(19) United States
(12) Patent Application Publication
(10) Pub. No.: US 2025/0082731 A1
(43) Pub. Date: Mar. 13, 2025

(54) TRANSDERMAL INSULIN FORMULATIONS AND METHODS OF USE THEREOF

(71) Applicant: Transdermal Delivery Solutions Corp. (d.b.a. HypoSpray Pharma), Palm Beach Gardens, FL (US)

(72) Inventors: Kenneth B. KIRBY, Palm Beach Gardens, FL (US); Gordon VON NEHRING, North Miami Beach, FL (US); Chandan A.S. ALAM, Hadley Wood (GB)

(73) Assignee: Transdermal Delivery Solutions Corp. (d.b.a. HypoSpray Pharma), Palm Beach Gardens, FL (US)

(21) Appl. No.: 18/028,036

(22) PCT Filed: Sep. 8, 2022
(86) PCT No.: PCT/US2022/042883

§ 371 Date: Mar. 23, 2023

Related U.S. Application Data
(60) Provisional application No. 63/346,022, filed on May 26, 2022.

(51) Int. Cl. A61K 38/28; A61K 9/00; A61K 9/08; A61K 47/08; A61K 47/10; A61K 47/14; A61K 47/20; A61K 47/22; A61K 47/44; A61P 3/10

(52) U.S. Cl. CPC A61K 38/28; A61K 9/0014; A61K 9/08; A61K 47/08; A61K 47/10; A61K 47/14; A61K 47/20; A61K 47/22; A61K 47/44; A61P 3/10

(57) ABSTRACT

Pharmaceutical formulations for the transdermal administration of insulin by topical application of the formulation to the skin of humans or other animals are described. Methodology for formulating such formulations which provide for very rapid uptake of the insulin and transmigration into and through the skin to either fatty tissues or the vascular system, while minimizing irritation to the skin and/or immunological response, is based on a transdermal delivery system wherein the insulin forms a true solution in a complex formed from particular solvents and solvent and solute modifiers in combination with skin stabilizers. Uptake of the medicament is further facilitated and made more rapid by including Forskolin or other source of cellular energy, namely induction of cAMP or cGMP.

### RELATED APPLICATIONS

This application claims benefit of priority of U.S. Provisional Patent Application No. 63/346,022, filed on May 26, 2022, which is incorporated herein by reference in its entirety.

### TECHNICAL FIELD

This disclosure relates to transdermal insulin formulations and methods for preparing, in addition to methods of administering such formulations.

### BACKGROUND

Insulin is a naturally occurring hormone secreted by the beta cells of the islands of Langerhans in the pancreas in response to increased levels of glucose in the blood. The hormone acts to regulate the metabolism of glucose and the processes associated with the intermediary metabolism of fat, carbohydrates and proteins. Insulin lowers blood glucose levels and promotes transport and entry of glucose into muscle cells and other tissues. Due to the chemical nature of insulin molecules, the traditional route of insulin administration in diabetic patients, who require multiple daily doses of insulin, is intradermal or subdermal injection

Efforts to develop a non-injectable transdermal insulin delivery system for the treatment of diabetes have not been successful to date.

While there have been attempts to develop transdermal “patches” or external pumps which contain a particular amount of insulin, which may be transferred at a particular rate, these patches and pumps have numerous limitations. One specific limitation is that insulin users must often gauge their requirements relative to physical activity and ingestion of carbohydrates. Additionally, there are different types of insulin, e.g., long-acting and short-acting, and the patient must develop skill in blending both the type and quantity of insulin in order to adequately control their blood sugar levels. The use of multiple patches having variable dosage strengths and insulin response characteristics thus becomes problematic.

Thus, there remains a long felt need for a transdermal delivery system for insulin in a convenient format and for a better means of delivering insulin to patients in need thereof.

### SUMMARY

The instant application discloses a transdermal insulin formulation.

In one example, the formulation comprises an insulin and can be present in an amount ranging from 0.001% to 3.5% (wt/wt) of the total formulation. In one example, the insulin is a rapid-acting insulin. In another example, the insulin is a short-acting insulin. In another example, the insulin is an intermediate-acting insulin. In another example, the insulin is a long-acting insulin. In another example, the insulin can be one or more selected from the group consisting of a rapid-acting insulin, a short-acting insulin, an intermediate-acting insulin, and a long-acting insulin

In another example, the formulation further comprises a solvent system.

In one example, the solvent system comprises two or more solvents.

In another example, the solvent system comprises at least one solvent modifier. In another example, the solvent system comprises at least one solute modifier.

In another example, the solvent system comprises at least one source of cellular activation energy.

In another example, the solvent system comprises at least one skin stabilizer.

In another example, the solvent system comprises two or more solvents, at least one solvent modifier, at least one source of cellular activation energy, and at least one skin stabilizer.

In another example, the solvent system comprises one or more ingredients selected from the group consisting of two or more solvents, at least one solvent modifier, at least one solute modifier, at least one source of cellular activation energy, and at least one skin stabilizer.

The two or more solvents can be selected from the group consisting of ethanol, ethylene glycol, propylene glycol, propylene carbonate, butylene glycol, acetone, and glycerol; and can be present in an amount ranging from 80% to 99% (wt/wt) of the total formulation.

The at least one solvent modifier can be selected from the group consisting of lemon oil (or/and d-limonene), Vitamin E, Pro-Vitamin B, D-panthenol, and methylsulfonylmethane (MSM); and can be present in an amount ranging from 0.0001% to 20% (wt/wt) of the total formulation.

The at least one solute modifier can be selected from the group consisting of terpenes, oxindole alkaloids, quercitrin (glycoside of quercetin), genistein and its glucoside, genistein, polyphenolic flavonoids, and other sugar adduct glucuronides; and can be present in an amount ranging from 0.003% to 5% (wt/wt) of the total formulation.

The at least one source of cellular activation energy can be selected from the group consisting of forskolin, colforsin, methylxanthines, Saikogenin and Saikosaponin, angelic acid, phellopterin, oxypeucedanin, acetylcholine, cytidine diphosphocholine, and ascorbic acid; and can be present in an amount ranging from 0.01% to 0.1% (wt/wt) of the total formulation.

The at least one skin stabilizer can be selected from the group consisting of glycerin monolaurate, Vitamin D3, alkoxy glycerols, eicosapentaenoic acid (EPA), docosahexaenoic acid (DHA), gamma-linolenic acid (GLA), Vitamin E, D-panthenol, phytantriol, dehydroepiandrosterone (DHEA), pregnenolone, pregnenolone acetate, esculin, allantoin, and ascorbyl palmitate; and can be present in an amount ranging from 0.05% to 5% (wt/wt) of the total formulation.

In another example, the solvent system comprises one or more ingredients selected from the group consisting of a membrane permeability modifier, an enzyme activator, and a capillary dilator.

In one example, the solvent system comprises ethanol, propylene carbonate, acetone, and phosphoric acid. In another example, the solvent system comprises ethanol, propylene glycol, acetone, and phosphoric acid. In another example, the solvent system comprises ethanol, propylene glycol, propylene carbonate, acetone, and phosphoric acid.

In another example, the solvent system comprises ethanol, propylene carbonate, acetone, lemon oil, Vitamin E, Phytantriol, Dexpanthenol, Lauricidin, methylsulfonylmethane (MSM), Forskolin, and phosphoric acid.

In another example, the phosphoric acid in an amount wherein the molecular ratio of phosphoric acid to the insulin ranges from 0.2 to 2. In another example, the formulation comprises 1.18 moles of phosphoric acid per mole of insulin.

In one example, the insulin comprised in a formulation described herein has a molecular weight ranging from 340 Daltons to 22,000 Daltons.

In another example, the insulin is human insulin.

In one example, molecular property of the insulin and the solvent system are substantially similar. The molecular property can be van der Waals forces or dipole moments.

In another example, the formulation comprises an insulin and a solvent system, wherein the solvent system comprises one or more selected from the group consisting of a solvent, a solvent modifier, a solute modifier, a source of cellular activation energy, and a skin stabilizer, and optionally, one or more selected from the group consisting of a membrane permeability modifier: an enzyme activator; and a capillary dilator, and wherein the insulin and the solvent system exhibit substantially similar van der Waals forces and/or dipole moments.

In one example, the formulation is a topical formulation.

In one example, the formulation is formulated in a liquid dosage form. In one example, the liquid dosage form can be in a range of 0.1 mL to 1 mL. In another example, the liquid dosage form comprises the at least one insulin in an amount ranging from 1 IU/mL to 1000 IU/mL of insulin.

In some examples, the dosage form of a transdermal insulin formulation disclosed herein includes liquid dosage forms, such as, for example solutions, liquid sprays, lotions, and the like.

In some examples, the dosage form of a transdermal insulin formulation disclosed herein can be applied to any area of skin, such as, forearm, upper arm, back, and chest.

In one example, the transdermal insulin formulation(s) as described herein are administered in a dosage ranging from 1 IU/day to 1000 IU/day of insulin.

In some examples, the transdermal insulin formulation(s) as described herein can be designed for fast release and transdermal absorption of insulin, or slow release and transdermal absorption of insulin over a prolonged period of time.

In one example, disclosed herein is a method for delivering insulin to a subject in need thereof, the method comprising administering to the subject a therapeutically effective amount of a formulation described herein.

In one example, disclosed herein is a method for stabilizing glucose levels in a subject receiving insulin, comprising administering to the subject in need thereof a therapeutically effective amount of a formulation described herein.

In one example, disclosed herein is a method for treating diabetes, comprising administering to the subject in need thereof a therapeutically effective amount of a formulation described herein.

In one example, disclosed herein is a method for reducing hypoglycemia, comprising administering to the subject in need thereof a therapeutically effective amount of a formulation described herein. In one example, disclosed herein is a method for delivering insulin while minimizing hypoglycemia, comprising administering to the subject in need thereof a therapeutically effective amount of a formulation described herein.

In one example, disclosed herein is a method of delivering 90% or more of the at least one insulin rapidly through the skin to the underlying fatty tissue Interstitium and/or capillary plexus. This delivery may be accomplished in only a few to several tens of seconds or just a few minutes or less.

In one example, disclosed herein is a transdermal insulin formulation for treatment of a living body by rapidly delivering an effective dose at least one insulin across the skin by application of said transdermal insulin formulation to an area of the skin, said transdermal insulin formulation comprising said at least one insulin and a solvent system, said at least one insulin having a molecular weight in excess of 300 Daltons, said at least one insulin having molecular properties including van der Waals forces and dipole moments, said at least one insulin dissolved in said solvent system as a solute, said solvent system having molecular properties including van der Waals forces and dipole moments, said molecular properties of said solvent system shifting the aggregate dielectric constant of the solute+solvent system to substantially the same or approximately ±20%.

The instant application also discloses a method for preparing a transdermal insulin formulation as described herein. In one example, the method comprises (a) selecting the at least one insulin, (b) determining an effective dose of said at least one insulin, said effective dose of said at least one insulin having molecular properties including van der Waals forces and dipole moments. (c) quantifying said molecular properties of said at least one insulin, (d) determining an amount of a solvent system to solubilize said effective dose of at least one insulin, said amount of the solvent system having molecular properties including van der Waals forces and dipole moments: (e) quantifying said molecular properties of said amount of the insulin, (f) comparing said molecular properties of the at least one insulin and said molecular properties of the solvent system, (g) determining that the molecular properties of the solvent system without solute are substantially the same or approximately ±20% as the molecular properties of the at least one insulin, and (h) combining said solvent system and said at least one insulin to provide the transdermal insulin formulation.

In one example, the method for preparing a transdermal insulin formulation as described herein comprises selecting one or more ingredients for the solvent system for determining an amount of a solvent system to solubilize said effective dose of at least one insulin, said one or more ingredients are selected from the group consisting of a solvent, a solvent modifier, a solute modifier, a source of cellular activation energy, a skin stabilizer, and a combination thereof: each of said one or more ingredients having distinct molecular properties including van der Waals forces and dipole moments.

In another example, the method for preparing a transdermal insulin formulation as described herein comprises selecting one or more ingredients for the solvent system for determining an amount of a solvent system to solubilize said effective dose of at least one insulin, said one or more ingredients are selected from the group consisting of a solvent, a solvent modifier, a solute modifier, a source of cellular activation energy, a skin stabilizer, one membrane permeability modifier, at least one enzyme activator, at least one capillary dilator and a combination thereof: each of said one or more ingredients having similar molecular properties including van der Waals forces and dipole moments.

In one example, disclosed herein is a method of selecting the ingredients and amounts to prepare a transdermal insulin formulation as described herein, wherein the method comprises the steps of: (a) selecting at least one insulin necessary to treat a specific condition, (b) quantifying the amount of said insulin for an effective dose, (c) quantifying the molecular properties of said insulin to include van der Waals forces and the dipole moments, (d) surveying solvents for said insulin, (e) quantifying the amounts of said solvents to solubilize said insulin, (f) quantifying the molecular properties of said solvents to include van der Waals forces and dipole-moments, (g) comparing the molecular properties of said solvents to said molecular properties of said insulin, (h) determining additional ingredients to form a solvent system for transmigration, (i) quantifying the molecular properties of said additional ingredients to include van der Waals forces and mol-moments, (j) determining a weighted sum of said molecular properties of said additional ingredients and said molecular properties of said solvents to determine molecular properties of said solvent system, (k) summing said molecular properties of said solvent system plus said insulin: (l) comparing (j) and (k), and (m) selecting said solvent system wherein said molecular properties of said at least one insulin in the solvent system are substantially the same as said molecular properties of said solvent system without the insulin.

In one example described herein, van der Waals forces and/or dipole-moments of the at least one insulin in the solvent system are approximately ±20% of van der Waals forces and/or dipole-moments of said solvent system without the insulin. In one example described herein, van der Waals forces and/or dipole-moments of the at least one insulin in the solvent system are approximately ±15% of van der Waals forces and/or dipole-moments of said solvent system. In one example described herein, van der Waals forces and/or dipole-moments of the at least one insulin in the solvent system are approximately ±10% of van der Waals forces and/or dipole-moments of said solvent system. In one example described herein, van der Waals forces and/or dipole-moments of the at least one insulin in the solvent system are approximately ±5% of van der Waals forces and/or dipole-moments of said solvent system.

### BRIEF DESCRIPTION OF THE DRAWINGS

FIG. 1 is a graph summarizing the glucose levels from Experiment 1 described herein.

FIG. 2 is a graph summarizing the glucose levels from Experiment 2 described herein.

FIG. 3 is a graph summarizing the glucose levels from Experiment 3 described herein.

FIG. 4 is a graph of the Subject's comparative response to Novolog® on days after dosing with a transdermal insulin formulation disclosed herein.

FIG. 5 is a histogram of the mean OD values for permeation of a transdermal insulin formulation disclosed herein, Injectable Insulin, or PBS control.

FIG. 6 is a histogram of the mean OD values for permeation of a transdermal insulin formulation disclosed herein vs. Injectable Insulin through Artificial Skin Model.

FIG. 7 depicts interpolated concentrations of permeation of transdermal formulation vs Injectable Insulin through Artificial Skin Model for each timepoint, plus the total recovered amount of Insulin.

FIG. 8 shows the graph for Insulin ELISA standard curve.

FIG. 9 shows immunohistochemical analysis of control fibroblasts and keratinocytes at time points 0 minutes (A), 5 minutes (B), 10 minutes (C), 20 minutes (D), 40 minutes (E), and 60 minutes (F).

FIG. 10 shows immunohistochemical analysis of fibroblasts and keratinocytes at time points 0 minutes (A), 5 minutes (B), 10 minutes (C), 20 minutes (D), 40 minutes (E), and 60 minutes (F) treated with injectable insulin.

FIG. 11 shows immunohistochemical analysis of fibroblasts and keratinocytes at time points 0 minutes (A), 5 minutes (B), 10 minutes (C), 20 minutes (D), 40 minutes (E), and 60 minutes (F) treated with transdermal insulin formulation as described herein.

FIG. 12 shows the blood glucose levels measured following 4.5 hours fasting, using Accu-Chek® active blood glucose meter and strips, before and after the application of 1 ml/kg of the vehicle (placebo) at 5, 10, 20, 40, 60, 90 and 120 minutes.

FIG. 13 shows the blood glucose levels measured following 4.5 hours fasting, using Accu-Chek® active blood glucose meter and strips, before and after the application of human insulin solution (0.1 IU/kg/ml) at 5, 10, 20, 40, 60, 90 and 120 minutes.

FIG. 14 shows the blood glucose levels measured following 4.5 hours fasting, using Accu-Chek® active blood glucose meter and strips, before and after the application of human insulin solution (0.2 IU/kg/ml) at 5, 10, 20, 40, 60, 90 and 120 minutes.

FIG. 15 shows the blood glucose levels measured following 4.5 hours fasting, using Accu-Chek® active blood glucose meter and strips, before and after the application of human insulin solution (0.4 IU/kg/ml) at 5, 10, 20, 40, 60, 90 and 120 minutes.

FIG. 16 shows the blood glucose levels measured following 4.5 hours fasting, using Accu-Chek® active blood glucose meter and strips, before and after the application of human insulin solution (0.8 IU/kg/ml) at 5, 10, 20, 40, 60, 90 and 120 minutes.

FIG. 17 shows the blood glucose levels measured following 4.5 hours fasting, using Accu-Chek® active blood glucose meter and strips, before and after the application of human insulin solution (1.6 IU/kg/ml) at 5, 10, 20, 40, 60, 90 and 120 minutes.

FIG. 18 shows data expressed as the percentage of variation in blood glucose levels relative to the basal levels (Time 0), after the transdermal application of human insulin solution (0.1 IU/kg/ml), or the vehicle (1 ml/kg).

FIG. 19 shows data expressed as the percentage of variation in blood glucose levels relative to the basal levels (Time 0), after the transdermal application of human insulin solution (0.2 IU/kg/ml), or the vehicle (1 ml/kg).

FIG. 20 shows data expressed as the percentage of variation in blood glucose levels relative to the basal levels (Time 0), after the transdermal application of human insulin solution (0.4 IU/kg/ml), or the vehicle (1 ml/kg).

FIG. 21 shows data expressed as the percentage of variation in blood glucose levels relative to the basal levels (Time 0), after the transdermal application of human insulin solution (0.8 IU/kg/ml), or the vehicle (1 ml/kg).

FIG. 22 shows data expressed as the percentage of variation in blood glucose levels relative to the basal levels (Time 0), after the transdermal application of human insulin solution (1.6 IU/kg/ml), or the vehicle (1 ml/kg).

FIG. 23 shows human insulin levels measured in cumulative plasma samples collected from time 0 (before treatment) to 120 min after treatment.

FIG. 24 shows a table of examples of transdermal insulin formulations described herein.

### DETAILED DESCRIPTION

### Definitions

The examples herein and the various features and advantageous details thereof are explained more fully with reference to the non-limiting examples that are illustrated in the accompanying drawings and detailed in the following description. Descriptions of well-known features and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Unless otherwise indicated, the definitions and embodiments described in this, and other sections are intended to be applicable to all embodiments and aspects of the present application herein described for which they are suitable as would be understood by a person skilled in the art.

The singular forms “a”, “an” and “the” as used herein include plural references unless the content clearly dictates otherwise. For example, an embodiment including “an agent” should be understood to present certain aspects with one compound or two or more additional compounds.

The terms “about”, “substantially”, and “approximately” as used herein mean a reasonable amount of deviation of the modified term such that the end result is not significantly changed. These terms of degree should be construed as including a deviation of at least ±5%, at least ±10%, at least ±15%, or at least ±20%, of the modified term if this deviation would not negate the meaning of the word it modifies.

The term “suitable” as used herein means that the selection of the compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “agent” and “ingredient” as used herein are interchangeable and indicate a compound or mixture of compounds that, when added to a formulation, tend to produce a particular effect on the formulation's properties.

The term “and/or” as used herein means that the listed items are present, or used, individually or in combination. In effect, this term means that “at least one of” or “one or more” of the listed items is used or present.

The term “delivery solution” as used herein refers to a liquid or semi-solid mixture of chemicals that can be broadly classified as solvents, solvent modifiers and/or the other chemical inclusions into which pharmaceutical active ingredients are stably dissolved to serve as a vehicle for the introduction of said active pharmaceutical ingredient(s) into physiology, whether by injection, ingestion or across the skin. The term “pharmaceutical active ingredient” in this context refers to insulins and other compounds used to treat diabetes.

The term “delivery system” as used herein refers to a “delivery solution” as described above formulated in particular ratios and ranges of ratios relative to each other and a value determined for the solution into which pharmaceutical active ingredients are stably dissolved in order serve as a vehicle for the introduction of said active pharmaceutical ingredient(s) into physiology, whether by injection, ingestion or across the skin.

The terms “formulation,” “composition,” “pharmaceutical formulation,” and “pharmaceutical composition” as used herein are interchangeable and refer to a formulation for pharmaceutical use.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the formulation in which it is contained. The term “pharmaceutically acceptable” also refer to being compatible with the treatment of animals, for example, humans.

The term “effective amount” as used herein means an amount sufficient to achieve the desired result and accordingly will depend on the ingredient and its desired result. Nonetheless, once the desired effect is known, determining the effective amount is within the skill of a person skilled in the art.

The term “treating”, and “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e., not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. Treatment methods comprise administering to a subject a therapeutically effective amount of a formulation as described herein and optionally consists of a single administration, or alternatively comprises a series of applications. The length of the treatment period depends on a variety of factors, such as the severity of the condition, the age of the patient, the concentration of active ingredient or agent, the activity of the formulations described herein, and/or a combination thereof. It will also be appreciated that the effective dosage of a formulation used for the treatment or prophylaxis may increase or decrease over the course of a particular treatment or prophylaxis regime. Changes in dosage may result and become apparent by standard diagnostic assays known in the art. In some instances, chronic administration may be required. For example, the formulations are administered to the subject in an amount and for duration sufficient to treat the patient.

The term “topical formulation” as used herein includes a formulation that is suitable for topical application to the skin. A topical formulation may, for example, be used to confer a therapeutic benefit to its user. Specific topical formulations can be used for local, regional, or transdermal application of substances.

The term “transdermal” as used herein includes a process that occurs through the skin. The terms “transdermal,” “percutaneous” and “transcutaneous” can be used interchangeably. In certain embodiments, “transdermal” also includes epicutaneous. Transdermal administration is often applied where systemic delivery of an active is desired, although it may also be useful for delivering an active to tissues underlying the skin with minimal systemic absorption.

The term “transdermal application” as used herein includes administration through the skin. Transdermal application can be used for systemic delivery of an active agent: however, it is also useful for delivery of an active agent to tissues underlying the skin with minimal systemic absorption. In certain embodiments, “transdermal application” can also include epicutaneous application.

The term “pharmaceutically acceptable salt” means an acid addition salt or basic addition salt which is suitable for or compatible with the treatment of subjects, including human subjects.

The term “diabetes” as used herein is intended to mean all diabetic conditions, including, without limitation, diabetes mellitus, genetic diabetes, type 1 diabetes, type 2 diabetes, type 3 diabetes, type 4 mature onset, type 5 Mature onset of the Young (MODY) and gestational diabetes. The term “diabetes” also refers to the chronic disease characterized by relative or absolute deficiency of insulin that results in glucose intolerance. Type 1 diabetes is also referred to as insulin dependent diabetes mellitus (IDDM) and also includes, for example, juvenile-onset diabetes mellitus. Type 1 is primarily due to the destruction of pancreatic β-cells. Type 2 diabetes mellitus is also known as non-insulin dependent diabetes mellitus (NIDDM) and is characterized, in part, by impaired insulin release following a meal. Insulin resistance can also be a factor leading to the occurrence of type 2 diabetes mellitus. Type 3 diabetes results from traumatic injury to the insulin-producing tissues resulting in cessation of or dramatic reduction of production of insulin. Type 4 diabetes is caused by insulin resistance in older people who don't have overweight or obesity. Type 5 diabetes or MODY 5 or genetic diabetes is a form of diabetes caused by a mutation of a single gene. The mutation causes pancreatic beta cells to function abnormally, leading to insufficient production of insulin. In some cases, insulin resistance develops. Gestational Diabetes occurs during pregnancy in response to hormonal changes in effect during gestation.

The term “diabetes” is also intended to include those individuals with hyperglycemia, including chronic hyperglycemia, hyperinsulinemia, impaired glucose homeostasis or tolerance, and insulin resistance. Plasma glucose levels in hyperglycemic individuals include, for example, glucose concentrations greater than normal as determined by reliable diagnostic indicators. Such hyperglycemic individuals are at risk or predisposed to developing overt clinical symptoms of diabetes mellitus.

Numerical ranges as used herein are intended to include every number and subset of numbers contained within that range, whether specifically disclosed or not. Further, these numerical ranges should be construed as providing support for a claim directed to any number or subset of numbers in that range.

It is understood that all ingredients used in the formulations of this invention must, within the applied and recommended dosages, be non-toxic and safe for human use. Also, all amounts, parts and percentages in the following description and appended claims are on a weight basis unless otherwise noted.

### Formulation

Disclosed herein is a transdermal insulin formulation comprising at least one insulin and a solvent system. Insulin potency varies from lot to lot and is defined by the unit IU (International Unit or Unite International) typically 28 IU/mg.

The at least one insulin can be selected from the group consisting of rapid-acting insulin, short-acting insulin, intermediate-acting insulin, long-acting insulin, and a mixture thereof. In another example, the at least one insulin is human insulin. The at least one insulin comprised in a formulation described herein can have a molecular weight ranging from 340 Daltons to 22,000 Daltons.

In some examples, the formulation contains 10 IU/ml insulin. In other examples, the formulation contains 50 IU/ml insulin. In yet other examples, the formulation contains 100 IU/ml insulin. In yet other examples, the formulation contains 200 IU/ml insulin. In yet other examples, the formulation contains 500 IU/ml insulin. As such, the inulin potency of the formulations described herein can be, for example, 10 IU/ml, 50 IU/ml, 100 IU/ml, 200 IU/ml, or 500 IU/ml.

In some examples, the formulation described herein is designed to be delivered in predetermined amounts. In some examples, the Delivery System delivers 0.2 mL to 1 mL and comprises an amount of insulin ranging from 7 to 1,700 IU/mL. In some examples, the formulation is prepared in a unit dosage form, wherein the volume of the unit dosage form ranges from 0.2 mL to 1 mL and wherein the unit dosage form comprises insulin in an amount ranging from 0.25 mg to 60 mg. In some examples, the volume of the unit dosage form is 0.2 mL, 0.3 mL, 0.4 mL, 0.5 mL, 0.6 mL, 0.7 mL, 0.8 mL, 0.9 mL, or 1.0 mL. In some examples the unit dosage form comprises insulin in an amount of 7 IU, 14 IU, 28 IU, 140 IU, 280 IU, 350 IU, 420 IU, 490 IU, 560 IU, 630 IU, 700 IU, 770 IU, 840 IU, 910 IU, 980 IU, 1,050 IU, 1,120 IU, 1,190 IU, 1,260 IU, 1,330 IU, 1,400 IU, 1,470 IU, 1,540 IU, 1,610 IU, or 1,680 IU. In some examples, the unit dosage form comprises insulin in an amount within a range of the amounts described in this paragraph.

In one example, the transdermal insulin formulation(s) as described herein are administered within a dose range of 1 IU/dose to 750 IU/dose of insulin, 40 IU/dose to 120 IU/dose of insulin, 0.5 IU/dose to about 5 IU/dose; and from 50 IU/dose to 500 IU/dose of insulin.

In some examples, the transdermal, the included insulin formulation(s) as described herein can be designed to deliver for fast release insulin. In other embodiments, the transdermal insulin formulation(s) as described herein can be designed for slow-release insulin effective over a prolonged period of time via transdermal absorption of.

Rapid-acting insulin covers insulin needs for meals eaten at the same time as the injection. Short-acting insulin covers insulin needs for meals eaten within 30-60 minutes. Intermediate-acting insulin covers insulin needs for about half the day or overnight. This type of insulin is often combined with a rapid- or short-acting type. Long-acting insulin covers insulin needs for about one full day. This type is often combined, when needed, with rapid- or short-acting insulin. Representative insulins are set out in Table 1 below:

TABLE 1
Insulin Forms and Brands

| Type of Insulin and | | | | | | |
|---|---|---|---|---|---|---|
| Brand Names | Onset | | Peak | | Duration | |
| Rapid-Acting | | | | | | |
| Aspart (Novolog ®) | 10-20 | min. | 40-50 | min. | 3-5 | hours |
| Glulisine (Apidra ®) | 20-30 | min. | 30-90 | min. | 1-2.5 | hours |
| Lispro (Humalog ®) | 15-30 | min. | 30-90 | min | 3-5 | hours |
| Short-Acting | | | | | | |
| Regular (R) or | 30 min.-1 hour | | 2-5 | hours | 5-8 | hours |
| Novolin | | | | | | |
| Velosulin (for | 30 min.-1 hour | | 1-2 | hours | 2-3 | hours |
| use in the insulin | | | | | | |
| pump) | | | | | | |
| Intermediate-Acting | | | | | | |
| NPH (N) | 1.5-2 | hours | 4-12 | hours | 18-24 | hours |
| Human Insulin | 1 | | 3-4 | hours | 8 | hours |
| (Humulin R) | | | | | | |
| Long-Acting | | | | | | |
| Humalog mix 75/25 | 15 | min. | 30 min.- | | 16-20 | hours |
| | | | 2 ½ hours | | | |
| Humulin 50/50 | 30 | min. | 2-5 | hours | 18-24 | hours |
| Humulin 70/30 | 30 | min. | 2-4 | hours | 14-24 | hours |

| | | | | | |
|---|---|---|---|---|---|
| Insulin detemir | 1-2 | hours | 6-8 | hours | Up to 24 hours |
| (Levemir ®) | | | | | |

| Insulin glargine | 1-1 ½ | hours | No peak time. | 20-24 | hours |
|---|---|---|---|---|---|
| (Basaglar ®, | | | | | |
| Lantus ®, Toujeo ®) | | | Insulin is | | |
| | | | delivered at a | | |
| | | | steady level. | | |
| Insulin degludec | 30-90 | min. | No peak time | 42 | hours |
| (Tresiba ®) | | | | | |

| | | | | | |
|---|---|---|---|---|---|
| Novolin 70/30 | 30 | min. | 2-12 | hours | Up to 24 hours |
| Novolog 70/30 | 10-20 | min. | 1-4 | hours | Up to 24 hours |

The at least one insulin can be selected from the group consisting of rapid-acting insulin, short-acting insulin, intermediate-acting insulin, long-acting insulin, and a mixture thereof; and can be present in an amount ranging from 0.1% to 25% (wt/wt) of the total formulation.

In one example, the transdermal insulin formulation as described herein comprises at least one insulin in an amount ranging from 0.1% to 25% (wt/wt) of the total formulation. In another example, the at least one insulin is present in an amount ranging from 0.1% to 20% (wt/wt) of the total formulation. In yet another example, the at least one insulin is present in an amount of 0.1% to 15% (wt/wt) of the total formulation. In yet another example, the at least one insulin is present in an amount ranging from 0.1% to 10% (wt/wt) of the total formulation. In yet another example, the at least one insulin is present in an amount ranging from 0.1% to 7.5% (wt/wt) of the total formulation. In yet another example, the at least one insulin is present in an amount ranging from 0.1% to 5% (wt/wt) of the total formulation. In yet another example, the at least one insulin is present in an amount ranging from 0.1% to 2.5% (wt/wt) of the total formulation. In yet another example, the at least one insulin is present in an amount ranging from 0.1% to 1% (wt/wt) of the total formulation. In yet another example, the at least one insulin is present in an amount ranging from 0.1% to 0.5% (wt/wt) of the total formulation. In yet another example, the at least one insulin is present in an amount ranging from 0.1% to 0.45% (wt/wt) of the total formulation. In yet another example, the at least one insulin is present in an amount ranging from 0.1% to 0.40% (wt/wt) of the total formulation. In yet another example, the at least one insulin is present in an amount ranging from 0.1% to 0.35% (wt/wt) of the total formulation. In yet another example, the at least one insulin is present in an amount ranging from 0.1% to 0.30% (wt/wt) of the total formulation. In yet another example, the at least one insulin is present in an amount ranging from 0.1% to 0.25% (wt/wt) of the total formulation. In yet another example, the at least one insulin is present in an amount ranging from 0.1% to 0.20% (wt/wt) of the total formulation.

In one example, the transdermal insulin formulation as described herein comprises at least one insulin in an amount of 0.1% (wt/wt) of the total formulation. In another example, the at least one insulin is present in an amount of 0.15% (wt/wt) of the total formulation. In yet another example, the at least one insulin is present in an amount of 0.20% (wt/wt) of the total formulation. In yet another example, the at least one insulin is present in an amount of 0.3% (wt/wt) of the total formulation. In yet another example, the at least one insulin is present in an amount of 0.4% (wt/wt) of the total formulation. In yet another example, the at least one insulin is present in an amount of 0.5% (wt/wt) of the total formulation. In yet another example, the at least one insulin is present in an amount of 0.6% (wt/wt) of the total formulation. In yet another example, the at least one insulin is present in an amount of 0.7% (wt/wt) of the total formulation. In yet another example, the at least one insulin is present in an amount of 0.8% (wt/wt) of the total formulation. In yet another example, the at least one insulin is present in an amount of 0.9% (wt/wt) of the total formulation. In yet another example, the at least one insulin is present in an amount of 1% (wt/wt) of the total formulation. In yet another example, the at least one insulin is present in an amount of 1.5% (wt/wt) of the total formulation. In yet another example, the at least one insulin is present in an amount of 2% (wt/wt) of the total formulation. In yet another example, the at least one insulin is present in an amount of 2.5% (wt/wt) of the total formulation. In yet another example, the at least one insulin is present in an amount of 5% (wt/wt) of the total formulation. In yet another example, the at least one insulin is present in an amount of 7.5% (wt/wt) of the total formulation. In yet another example, the at least one insulin is present in an amount of 10% (wt/wt) of the total formulation. In another example, the at least one insulin is present in an amount of 12.5% (wt/wt) of the total formulation. In yet another example, the at least one insulin is present in an amount of 15% (wt/wt) of the total formulation. In yet another example, the at least one insulin is present in an amount of 17.5% (wt/wt) of the total formulation. In yet another example, the at least one insulin is present in an amount of 20% (wt/wt) of the total formulation. In yet another example, the at least one insulin is present in an amount of 22.5% (wt/wt) of the total formulation. In yet another example, the at least one insulin is present in an amount of 25% (wt/wt) of the total formulation. In yet another example, the at least one insulin is present in an amount within a range of the amounts mentioned in this paragraph.

### Solvent System

Disclosed herein are transdermal insulin formulation(s) comprising at least one insulin and a solvent system, wherein the solvent system comprises one or more ingredients selected from the group consisting of two or more solvents, at least one solvent modifier, at least one solute modifier, at least one source of cellular activation energy, and at least one skin stabilizer

### Solvents

The solvent is the principal component of the carrier for insulin and, preferably, is one in which insulin is soluble or at least substantially soluble or can be made soluble or become more soluble, by addition of one or more solvent modifying agents. As used herein, by “substantially soluble” is meant that the minimum effective dose of insulin, generally at least 0.25 mg, preferably at least 0.5 mg, ideally at least 1 mg, will dissolve in 1 mL of the solvent(s) or in 1 mL of a mixture of the solvent(s) with solvent modifying agent(s).

Preferred solvents include lower alcohols of from about 2 to about 6 carbon atoms, preferably from 2 to 4 carbon atoms and may be monoalcohols, such as, for example, ethanol, isopropanol, sec-butanol, or polyols, such as, for example, ethylene glycol, propylene glycol, propylene carbonate, butylene glycol, glycerol. Mixtures of solvents may be used. Other solvents, such as ketones, e.g., acetone, methyl ethyl ketone, ethers, e.g., ethyl ether, may also be used, in amounts which will be safe and non-toxic in use.

While the solvent system is generally non-aqueous, water may also be introduced as a component of one of the other ingredients, for example, as an alcohol: water azeotrope, etc. When water is present in the solvent it will usually constitute less than about 50 percent, preferably less than about 10 percent, especially, preferably, less than about 2 percent, by weight of the total solvent although more or less may be used. Furthermore, as will become apparent by the examples to follow, the formulations disclosed in this application and utilizing the principles which will be described in more detail, hereinafter, may also be formulated as aqueous emulsions, including wherein the aqueous phase is the major and continuous phase. Such aqueous emulsions, as is the case with non-aqueous (usually less than about 5%, especially less than about 2%, of water) solvent systems, will be rapidly absorbed by and release insulin, in less than one minute.

The total amount of solvent(s) can be selected to assure dissolution of insulin and other additives and provide suitable product viscosity. The amount of solvent(s) falling within the range of from 5% wt/wt to 90% wt/wt, preferably from 25% wt/wt to 75% wt/wt, based on the total weight of the formulation, can be used.

### Solvent Modifiers

A solvent modifier for an insulin delivery system such as herein proposed is selected to modify the polarity of the solvent. The solvent modifier or mixture of solvent modifiers enables the solvent system [comprising solvent(s) and solvent modifier(s)] to form a weak complex with insulin, i.e., an association via van der Waals forces, thus yielding a stable formulation with a high insulin/solvent ratio. As used herein, “stable” is intended to have its normal and usual meaning, namely, that the formulation may be stored at room or elevated temperature for one or more days, usually 30 or more days, without undergoing phase separation. By “high insulin/solvent” ratio is meant at least 50 IU insulin per mL of solvent (or solvent plus modifying agents) and more generally, often amounts of insulin exceeding the solubility of the insulin in the solvent alone, or in each solvent of a multi-solvent system.

One or more of lemon oil (or/and d-limonene), Vitamin E, Pro-Vitamin B, D-panthenol and methylsulfonylmethane (MSM) can be used as a solvent modifier in the transdermal insulin formulation described herein.

The amount of solvent modifier can be selected to result in the desired insulin/solvent ratio, and depends on various factors, including, for example, primarily, the polarities, and polarizabilities, dipole moments, van der Waals forces of each component, including the solvent, solvent modifier, and insulin.

In this regard, in order to balance the polarities, dipole moments, of insulin to that of the solvent system the amount of the individual components of the solvent system can be selected such that the weighted (molar) average of the dipole moments of the individual components will be substantially the same in an empty system as the dipole moment of the solution with insulin dissolved.

The suitable amount of solvent modifier(s) to achieve the desired insulin/solvent ratio can be within the range of from 0.0001% wt/wt to 50% wt/wt, preferably, from 0.1% wt/wt to 35% wt/wt, more preferably, from 0.1% wt/wt to 5% wt/wt, based on the total weight of the formulation.

### Solute Modifiers

The solute modifier can be included in the formulation of the transdermal insulin formulation to facilitate dissolution of insoluble or sparingly soluble insulin at higher concentrations. Solute modifiers which form reversible or temporary complexes with insulin to facilitate passage through the skin while minimizing immunological response are especially effective. The solute modifier can also, optimally, be a nutritional compound which can be metabolized by the body once insulin is released from the complex.

Examples of solute modifiers include terpenes, oxindole alkaloids, quercitrin (glycoside of quercetin), genistein and its glucoside, genistein, polyphenolic flavonoids, and other sugar adduct glucoronides, such as, scutellarin, trans-ferulic acid, alpha-lipoic acid, sterols, such as, for example, cholesterol and cholesterol-like compounds and hormones, such as isoflavones, 3,3′-thiodipropionic acid (sulfonated propionic acid), phosphatidyl serine and choline, Vitamin D3, Vitamin K1, dehydroepiandrosterone (DHEA). Still other suitable candidate compounds include, for example, berberine, piper nigrum (e.g., Bioperine®), phosphatidyl serine, phosphatidyl choline. Another group of candidate compounds include boswellic acid, hypericum, and phytic acid.

The selection of a particular solute modifier will facilitate movement of insulin-complex past the stratum corneum and viable skin to its optimal targeted internal circulation system of Interstitium, blood, or lymph.

Suitable amount of the solute modifier can be determined based on such factors as, for example, solubility of the modifier in the system (e.g. solvent plus solvent modifiers), its molecular compatibility with insulin, its ability to modify the polarizability of insulin to increase the concentration (solubility) of insulin in the solvent, etc. The amount of solute modifier can range from 0.003% to 5%, preferably from 0.1% to 5%, more preferably from 0.1% to 4%, based on the weight of the total formulation. The amount of solute modifier or modifiers can be such that it is equivalent to the amount of insulin to provide a 1:1 interaction between modifier(s):insulin.

The above-described modifying agents, i.e., solvent and solute modifiers, as well as other components of solvent/carrier delivery system can be selected from substances which the body recognizes as usable building blocks of other physiological systems. This selection therefore facilitates nearly complete disassociation of insulin from the delivery system once in the body. Since these carrier/complex compounds are reducible to elemental building blocks of physiology they should do no harm to the body.

### Source of Cellular Activation Energy

A transdermal insulin formulation as described herein includes a source of cellular activation energy which serves the purpose of inducing high concentrations of enzyme-substrate complexes to be formed, such as by activation of the N, (stimulatory) protein of adeny late cyclase, thereby resulting in cellular levels of adenosine 3′,5′-cyclic monophosphate (cAMP) approaching the maximal limits of cellular cAMP concentration.

One example of such an agent includes extracts of the plant Coleus Forskohlii, and especially, Forskolin, a labdane diterpenoid. Other extracts of Coleus Forskohlii, such as, Colforsin or Coleonol, for example, can also be used.

Other examples of activation energy sources for stimulating generation of cAMP, either via precursors or cellular activators, include, for example, methyl xanthines, Saikogenin and Saikosaponin, Angelicae dahuricae radix (yielding angelic acid), phellopterin, oxypeucedanin.

Examples of substances which stimulate cellular production of cGMP can also be used and can be selected from the group consisting of acetylcholine, cytidine diphosphocholine, and ascorbic acid (vitamin C).

The amount of the source of cellular activation energy depends on such factors as, for example, the mechanism of action of insulin, energy of activation (positive or negative) when insulin encounters its receptor (to enhance or decrease cAMP or cGMP levels), etc. Suitable amounts of forskolin or acetylcholine or other source of cellular activation energy, can range from 0.001% to 0.1%, preferably, from 0.001% to 0.01%, more preferably, from 0.001% to 0.005%, based on the total weight of the formulation.

### Skin Stabilizers

Skin stabilizers can be included in a transdermal insulin formulation as described herein to stabilize the skin prior to passage and to assist the skin to repair any damage resulting from the transmigration of insulin and solvent and other components of the formulation.

Examples of substances that function as skin stabilizers and can be included in a formulation as described herein include glycerin monolaurate (e.g., as LauricidinR) and similar fatty acid esters, Vitamin D3, alkoxy glycerols, unsaturated fatty acids, such as, eicosapentaenoic acid (EPA), docosahexaenoic acid (DHA), and gamma-linolenic acid (GLA), Vitamin E (alpha tocopherol acetate) and the esters, e.g., acetate, and derivatives thereof, e.g., tocotrienol, D-panthenol, phytantriol, dehydroepiandrosterone (DHEA), pregnenolone, pregnenolone acetate, esculin, allantoin, ascorbyl palmitate, and the like.

Suitable amounts of the skin stabilizers can be determined based on such factors as, for example, type of reaction between insulin and skin, between solvent and skin, etc. Amounts of skin stabilizer, when present, can be 0.01% in an example of the formulation described herein.

In addition, the skin stabilizers can be present in an amount ranging from 0.05% to 5%, preferably, from 0.1% to 5%, more preferably, from 0.1% to 2%, by weight, based on total formulation. It is preferred to select stabilizers which will be effective in stabilizing the skin at as low a concentration as possible.

### Other Ingredients

### Enzyme Activators/Signaling Compounds

In one example, a transdermal insulin formulation as described herein can include enzyme activators/signaling compounds such as Forskolin and sulforaphane.

Suitable amounts of such enzyme activators/signaling compounds can range from 0.01% to 0.05%, preferably, from 0.01% to 0.02%, by weight, based on the total formulation.

Disclosed herein is a transdermal insulin formulation comprising at least one insulin and a solvent system.

In one example disclosed herein, the transdermal insulin formulation comprises at least one insulin and a solvent system, wherein the solvent system without Insulin comprises molecular properties substantially similar to the molecular properties of the solute in the system. In another example, the transdermal insulin formulation comprises at least one insulin and a solvent system, wherein the solvent system without insulin comprises molecular properties approximately +20% molecular properties of the solute in solution. In another example, the transdermal insulin formulation comprises at least one insulin and a solvent system, wherein the solvent system without insulin comprises molecular properties approximately +15% molecular properties of the solute in solution. In another example, the transdermal insulin formulation comprises at least one insulin and a solvent system without insulin, wherein the solvent system comprises molecular properties approximately #10% molecular properties of the solute in solution. In another example, the transdermal insulin formulation comprises at least one insulin and a solvent system, wherein the solvent system without insulin comprises molecular properties approximately +5% molecular properties of the solute in solution.

The molecular properties can be selected from van der Waals forces and/or dipole moments.

In this regard, it is understood that the dipole moment of a given compound may be taken directly from the literature, when available, or otherwise measured or calculated by standard techniques, including commercially available chemical modeling software packages. Generally, dipole moment is experimentally determined for an element or compound by suspending a molecule in an electromagnetic field and measuring the amount of energy (torque) to rotate the molecule one rotation. Dipole moment is correlated to van der Waals forces and the number of hydrogen bonds as well as electrostatic energy of a molecule. Two chemical entities with approximately the same dipole moment will usually have an affinity for and be attracted to one another without the necessity for covalent bonding.

To determine the dipole moment of the solvents(s) and modifier(s), a weighted average of the-dipole moments of the individual components is used. The weighted average should closely approximate the dipole moment of the solute. The closer the match the faster will be the rate of transmigration through the skin. The Delivery System will be modified, as necessary, to move the dipole moment of the system with modifying agents and other additives, including the solute, to as close as possible to that of the Delivery System without insulin, preferably within 15%, especially within 10%, most especially within 5%, of the dipole moment of the solute.

More specifically, in accordance with the preferred method for forming the formulations described herein, especially for increasing the amount of insulin which can be stably carried in solution in the transdermal delivery formulations described herein, the selection of and the amounts of the ingredients of the solvent system and other functional additives may be determined, in the first instance, by balancing the dipole moment of insulin relative to the dipole moment of the final formulation. The dipole moment of the final formulation is taken to be the weighted average dipole moments of each individual ingredient. The weighted average is obtained by calculating the sum of the dipole moments of the ingredients, where the dipole moment is obtained by multiplying the amount, in moles, of an ingredient, in a given volume, e.g., 100 cc, by the dipole moment for that ingredient. For purpose of this calculation, it is assumed that each ingredient in the formulation acts independently of the other ingredients. Thus, for example, the dipole moment of any particular ingredient does not take into account the electronic, e.g., repulsive or attractive, effects of other ingredients. However, by taking concentrations into consideration, that is, by multiplying individual dipole moments by molar concentrations, a reasonable approximation of the matching of the system's properties to balance for insulin will be achieved.

As in the case for balancing of dipole moments, as described herein, the formulation of the solvent system for insulin may be balanced for mole-van der Waals forces, when the insulin added to the solvent system, as a predictor of solubility of the desired amount(s) of insulin by bringing the sum of the mole-van der Waals forces for the solvent system with insulin to within +20%, preferably within +15%, especially preferably within +10%, and most especially preferably within +5%, of the sum of the mole-van der Waals forces of the solvent system without insulin.

When the difference between the sum of the mole-van der Waals forces of the solvent system plus insulin is greater than about 20%, especially greater than about 15% of the sum of mole-van der Waals forces for the solvent system without insulin, the desired amount of insulin will tend to be insoluble in the solvent system or may precipitate from solution upon standing overnight.

The transdermal insulin formulation as described herein provides for delivery at least about 90% or more of the at least one insulin rapidly through the skin to the underlying fatty tissue. This delivery may be accomplished in only a few to several tens of seconds or just a few minutes or less.

The solvent system comprises one or more ingredients selected from the group consisting of at least two solvents, at least one solvent modifier, at least one solute modifier, at least one source of cellular activation energy, at least one skin stabilizer, at least one membrane permeability modifier, at least one enzyme activator, and at least one capillary dilator.

In a preferred example, the solvent system comprises one or more ingredients selected from the group consisting of at least two solvents, at least one solvent modifier, at least one solute modifier, at least one source of cellular activation energy, and at least one skin stabilizer.

In a more preferred example, the solvent system comprises one or more ingredients selected from the group consisting of at least two solvents, at least one solvent modifier, at least one source of cellular activation energy, and at least one skin stabilizer.

In one example, disclosed herein is a transdermal formulation comprising at least one insulin and a solvent system, wherein the solvent system comprises two solvents, a solvent modifier, a source of cellular activation energy, and a skin stabilizer.

In one example, solvent can comprise one or more ingredients selected from the group consisting of ethanol, isopropanol, ethylene glycol, propylene carbonate, propylene glycol, acetone, and methyl ethyl ketone. In a preferred example, the solvent comprises one or more ingredients selected from the group consisting of ethanol, propylene carbonate, propylene glycol, and acetone. In a more preferred embodiment, the solvent comprises ethanol, propylene carbonate, and acetone.

In one example, the solvent system comprises ethanol in an amount of 35% (wt/wt) of the total formulation. In another example, the solvent system comprises ethanol in an amount of 36% (wt/wt) of the total formulation. In yet another example, the solvent system comprises ethanol in an amount of 37% (wt/wt) of the total formulation. In yet another example, the solvent system comprises ethanol in an amount of 38% (wt/wt) of the total formulation. In yet another example, the solvent system comprises ethanol in an amount of 39% (wt/wt) of the total formulation. In yet another example, the solvent system comprises ethanol in an amount of 40% (wt/wt) of the total formulation. In yet another example, the solvent system comprises ethanol in an amount of 41% (wt/wt) of the total formulation. In yet another example, the solvent system comprises ethanol in an amount of 42% (wt/wt) of the total formulation. In yet another example, the solvent system comprises ethanol in an amount of 43% (wt/wt) of the total formulation. In yet another example, the solvent system comprises ethanol in an amount of 44% (wt/wt) of the total formulation. In yet another example, the solvent system comprises ethanol in an amount of 45% (wt/wt) of the total formulation. In yet another example, the solvent system comprises ethanol in an amount of 46% (wt/wt) of the total formulation. In yet another example, the solvent system comprises ethanol in an amount of 47% (wt/wt) of the total formulation. In yet another example, the solvent system comprises ethanol in an amount of 48% (wt/wt) of the total formulation. In yet another example, the solvent system comprises ethanol in an amount of 49% (wt/wt) of the total formulation. In yet another example, the solvent system comprises ethanol in an amount of 50% (wt/wt) of the total formulation. In yet another example, the solvent system comprises ethanol in an amount within a range of the amounts described in this paragraph.

In one example, the solvent system comprises propylene carbonate in an amount of 40% (wt/wt) of the total formulation. In another example, the solvent system comprises propylene carbonate in an amount of 41% (wt/wt) of the total formulation. In yet another example, the solvent system comprises propylene carbonate in an amount of 42% (wt/wt) of the total formulation. In yet another example, the solvent system comprises propylene carbonate in an amount of 43% (wt/wt) of the total formulation. In yet another example, the solvent system comprises propylene carbonate in an amount of 44% (wt/wt) of the total formulation. In yet another example, the solvent system comprises propylene carbonate in an amount of 45% (wt/wt) of the total formulation. In yet another example, the solvent system comprises propylene carbonate in an amount of 46% (wt/wt) of the total formulation. In yet another example, the solvent system comprises propylene carbonate in an amount of 47% (wt/wt) of the total formulation. In yet another example, the solvent system comprises propylene carbonate in an amount of 48% (wt/wt) of the total formulation. In yet another example, the solvent system comprises propylene carbonate in an amount of 49% (wt/wt) of the total formulation. In yet another example, the solvent system comprises propylene carbonate in an amount of 50% (wt/wt) of the total formulation. In yet another example, the solvent system comprises propylene carbonate in an amount of 51% (wt/wt) of the total formulation. In yet another example, the solvent system comprises propylene carbonate in an amount of 52% (wt/wt) of the total formulation. In yet another example, the solvent system comprises propylene carbonate in an amount of 53% (wt/wt) of the total formulation. In yet another example, the solvent system comprises propylene carbonate in an amount of 54% (wt/wt) of the total formulation. In yet another example, the solvent system comprises propylene carbonate in an amount of 55% (wt/wt) of the total formulation. In yet another example, the solvent system comprises propylene carbonate in an amount within a range of the amounts described in this paragraph.

In one example, the solvent system comprises propylene glycol in an amount of 40% (wt/wt) of the total formulation. In another example, the solvent system comprises propylene glycol in an amount of 41% (wt/wt) of the total formulation. In yet another example, the solvent system comprises propylene glycol in an amount of 42% (wt/wt) of the total formulation. In yet another example, the solvent system comprises propylene glycol in an amount of 43% (wt/wt) of the total formulation. In yet another example, the solvent system comprises propylene glycol in an amount of 44% (wt/wt) of the total formulation. In yet another example, the solvent system comprises propylene glycol in an amount of 45% (wt/wt) of the total formulation. In yet another example, the solvent system comprises propylene glycol in an amount of 46% (wt/wt) of the total formulation. In yet another example, the solvent system comprises propylene glycol in an amount of 47% (wt/wt) of the total formulation. In yet another example, the solvent system comprises propylene glycol in an amount of 48% (wt/wt) of the total formulation. In yet another example, the solvent system comprises propylene glycol in an amount of 49% (wt/wt) of the total formulation. In yet another example, the solvent system comprises propylene glycol in an amount of 50% (wt/wt) of the total formulation. In yet another example, the solvent system comprises propylene glycol in an amount of 51% (wt/wt) of the total formulation. In yet another example, the solvent system comprises propylene glycol in an amount of 52% (wt/wt) of the total formulation. In yet another example, the solvent system comprises propylene glycol in an amount of 53% (wt/wt) of the total formulation. In yet another example, the solvent system comprises propylene glycol in an amount of 54% (wt/wt) of the total formulation. In yet another example, the solvent system comprises propylene glycol in an amount of 55% (wt/wt) of the total formulation. In vet another example, the solvent system comprises propylene glycol in an amount within a range of the amounts described in this paragraph.

In one example, the solvent system comprises acetone in an amount of 0.5% (wt/wt) of the total formulation. In another example, the solvent system comprises acetone in an amount of 0.75% (wt/wt) of the total formulation. In yet another example, the solvent system comprises acetone in an amount of 1.0% (wt/wt) of the total formulation. In yet another example, the solvent system comprises acetone in an amount of 1.25% (wt/wt) of the total formulation. In yet another example, the solvent system comprises acetone in an amount of 1.5% (wt/wt) of the total formulation. In yet another example, the solvent system comprises acetone in an amount of 1.75% (wt/wt) of the total formulation. In yet another example, the solvent system comprises acetone in an amount of 2.0% (wt/wt) of the total formulation. In yet another example, the solvent system comprises acetone in an amount of 2.25% (wt/wt) of the total formulation. In yet another example, the solvent system comprises acetone in an amount of 2.5% (wt/wt) of the total formulation. In yet another example, the solvent system comprises acetone in an amount of 2.75% (wt/wt) of the total formulation. In yet another example, the solvent system comprises acetone in an amount of 3.0% (wt/wt) of the total formulation. In yet another example, the solvent system comprises acetone in an amount of 3.25% (wt/wt) of the total formulation. In yet another example, the solvent system comprises acetone in an amount of 3.5% (wt/wt) of the total formulation. In yet another example, the solvent system comprises acetone in an amount of 3.75% (wt/wt) of the total formulation. In yet another example, the solvent system comprises acetone in an amount of 4.0% (wt/wt) of the total formulation. In yet another example, the solvent system comprises acetone in an amount of 4.25% (wt/wt) of the total formulation. In yet another example, the solvent system comprises acetone in an amount of 4.5% (wt/wt) of the total formulation. In yet another example, the solvent system comprises acetone in an amount of 4.75% (wt/wt) of the total formulation. In yet another example, the solvent system comprises acetone in an amount of 5.0% (wt/wt) of the total formulation. In yet another example, the solvent system comprises acetone in an amount within a range of the amounts described in this paragraph.

In some examples, the solvent system comprises an acid selected from the group consisting of an Arrhenius acid, a mineral acid, an organic acid, a Bronsted-Lowry acid, a strong acid, a weak acid, a diprotic acid and a triprotic acid. Examples of such acids include, but not limited to Hydrochloric acid, Phosphoric acid, Perchloric acid, Sulfuric acid, Nitric acid, Hydroiodic Acid, Lactic acid, Oxalic acid, Succinic acid, Hydrobromic acid, Nitrous acid & Ammonium Ion, Fluorosulfuric acid, Triflic acid, Fluroantimonic acid, Formic acid, Sulfurous acid, Benzoic acid, Carbonic acid, Citric acid, and Arsenic acid. The solvent system described herein can comprise an acid in an amount ranging from 0.25% (wt/wt) to 3.0% (wt/wt).

In one example, the solvent system comprises phosphoric acid in an amount of 0.25% (wt/wt) of the total formulation. In another example, the solvent system comprises phosphoric acid in an amount of 0.5% (wt/wt) of the total formulation. In yet another example, the solvent system comprises phosphoric acid in an amount of 0.75% (wt/wt) of the total formulation. In yet another example, the solvent system comprises phosphoric acid in an amount of 1.0% (wt/wt) of the total formulation. In yet another example, the solvent system comprises phosphoric acid in an amount of 1.25% (wt/wt) of the total formulation. In yet another example, the solvent system comprises phosphoric acid in an amount of 1.5% (wt/wt) of the total formulation. In yet another example, the solvent system comprises phosphoric acid in an amount of 1.75% (wt/wt) of the total formulation. In yet another example, the solvent system comprises phosphoric acid in an amount of 2.0% (wt/wt) of the total formulation. In yet another example, the solvent system comprises phosphoric acid in an amount of 2.25% (wt/wt) of the total formulation. In yet another example, the solvent system comprises phosphoric acid in an amount of 2.5% (wt/wt) of the total formulation. In yet another example, the solvent system comprises phosphoric acid in an amount of 2.75% (wt/wt) of the total formulation. In yet another example, the solvent system comprises phosphoric acid in an amount of 3.0% (wt/wt) of the total formulation. In yet another example, the solvent system comprises phosphoric acid in an amount within a range of the amounts described in this paragraph.

In one example, the solvent system comprises phosphoric acid, wherein molecular ratio of phosphoric acid to insulin is 0.2. In another example, the solvent system comprises phosphoric acid, wherein molecular ratio of phosphoric acid to insulin is 0.25. In another example, the solvent system comprises phosphoric acid, wherein molecular ratio of phosphoric acid to insulin is 0.3. In another example, the solvent system comprises phosphoric acid, wherein molecular ratio of phosphoric acid to insulin is 0.35. In yet another example, the solvent system comprises phosphoric acid, wherein molecular ratio of phosphoric acid to insulin is 0.4. In another example, the solvent system comprises phosphoric acid, wherein molecular ratio of phosphoric acid to insulin is 0.45. In yet another example, the solvent system comprises phosphoric acid, wherein molecular ratio of phosphoric acid to insulin is 0.5. In yet another example, the solvent system comprises phosphoric acid, wherein molecular ratio of phosphoric acid to insulin is 0.6. In yet another example, the solvent system comprises phosphoric acid, wherein molecular ratio of phosphoric acid to insulin is 0.7. In yet another example, the solvent system comprises phosphoric acid, wherein molecular ratio of phosphoric acid to insulin is 0.8. In yet another example, the solvent system comprises phosphoric acid, wherein molecular ratio of phosphoric acid to insulin is 0.9. In yet another example, the solvent system comprises phosphoric acid, wherein molecular ratio of phosphoric acid to insulin is 1.0. In yet another example, the solvent system comprises phosphoric acid, wherein molecular ratio of phosphoric acid to insulin is 1.1. In another example, the solvent system comprises phosphoric acid, wherein molecular ratio of phosphoric acid to insulin is 0.15. In yet another example, the solvent system comprises phosphoric acid, wherein molecular ratio of phosphoric acid to insulin is 1.2. In another example, the solvent system comprises phosphoric acid, wherein molecular ratio of phosphoric acid to insulin is 1.25. In yet another example, the solvent system comprises phosphoric acid, wherein molecular ratio of phosphoric acid to insulin is 1.3. In another example, the solvent system comprises phosphoric acid, wherein molecular ratio of phosphoric acid to insulin is 1.35. In yet another example, the solvent system comprises phosphoric acid, wherein molecular ratio of phosphoric acid to insulin is 1.4. In another example, the solvent system comprises phosphoric acid, wherein molecular ratio of phosphoric acid to insulin is 1.45. In yet another example, the solvent system comprises phosphoric acid, wherein molecular ratio of phosphoric acid to insulin is 1.5. In another example, the solvent system comprises phosphoric acid, wherein molecular ratio of phosphoric acid to insulin is 1.55. In yet another example, the solvent system comprises phosphoric acid, wherein molecular ratio of phosphoric acid to insulin is 1.6. In another example, the solvent system comprises phosphoric acid, wherein molecular ratio of phosphoric acid to insulin is 1.65. In yet another example, the solvent system comprises phosphoric acid, wherein molecular ratio of phosphoric acid to insulin is 1.7. In another example, the solvent system comprises phosphoric acid, wherein molecular ratio of phosphoric acid to insulin is 1.75. In yet another example, the solvent system comprises phosphoric acid, wherein molecular ratio of phosphoric acid to insulin is 1.8. In another example, the solvent system comprises phosphoric acid, wherein molecular ratio of phosphoric acid to insulin is 1.85. In yet another example, the solvent system comprises phosphoric acid, wherein molecular ratio of phosphoric acid to insulin is 1.9. In another example, the solvent system comprises phosphoric acid, wherein molecular ratio of phosphoric acid to insulin is 1.95. In yet another example, the solvent system comprises phosphoric acid, wherein molecular ratio of phosphoric acid to insulin is 2.0. In yet another example, the solvent system comprises phosphoric acid, wherein molecular ratio of phosphoric acid to insulin is within a range of the ratios described in this paragraph.

In one example, the solvent system comprises a solvent modifier in an amount of 0.001% (wt/wt) of the total formulation. In another example, the solvent system comprises a solvent modifier in an amount of 0.0025% (wt/wt) of the total formulation. In yet another example, the solvent system comprises a solvent modifier in an amount of 0.005% (wt/wt) of the total formulation. In yet another example, the solvent system comprises a solvent modifier in an amount of 0.01% (wt/wt) of the total formulation. In yet another example, the solvent system comprises a solvent modifier in an amount of 0.025% (wt/wt) of the total formulation. In yet another example, the solvent system comprises a solvent modifier in an amount of 0.05% (wt/wt) of the total formulation. In yet another example, the solvent system comprises a solvent modifier in an amount of 0.075% (wt/wt) of the total formulation. In yet another example, the solvent system comprises a solvent modifier in an amount of 0.1% (wt/wt) of the total formulation. In yet another example, the solvent system comprises a solvent modifier in an amount of 0.25% (wt/wt) of the total formulation. In yet another example, the solvent system comprises a solvent modifier in an amount of 0.5% (wt/wt) of the total formulation. In yet another example, the solvent system comprises a solvent modifier in an amount of 0.75% (wt/wt) of the total formulation. In yet another example, the solvent system comprises a solvent modifier in an amount of 1.0% (wt/wt) of the total formulation. In yet another example, the solvent system comprises a solvent modifier in an amount within a range of the amounts described in this paragraph. The solvent modifier can be one or more selected from the group consisting of lemon oil, Vitamin E, and methylsulfonylmethane (MSM).

In one example, the solvent system comprises a Forskolin in an amount of 0.001% (wt/wt) of the total formulation. In another example, the solvent system comprises Forskolin in an amount of 0.0025% (wt/wt) of the total formulation. In yet another example, the solvent system comprises Forskolin in an amount of 0.005% (wt/wt) of the total formulation. In yet another example, the solvent system comprises Forskolin in an amount of 0.01% (wt/wt) of the total formulation. In yet another example, the solvent system comprises Forskolin in an amount of 0.015% (wt/wt) of the total formulation. In yet another example, the solvent system comprises Forskolin in an amount of 0.02% (wt/wt) of the total formulation. In yet another example, the solvent system comprises Forskolin in an amount of 0.025% (wt/wt) of the total formulation. In yet another example, the solvent system comprises Forskolin in an amount of 0.03% (wt/wt) of the total formulation. In yet another example, the solvent system comprises Forskolin in an amount of 0.035% (wt/wt) of the total formulation. In yet another example, the solvent system comprises Forskolin in an amount of 0.04% (wt/wt) of the total formulation. In yet another example, the solvent system comprises Forskolin in an amount of 0.045% (wt/wt) of the total formulation. In yet another example, the solvent system comprises Forskolin in an amount of 0.05% (wt/wt) of the total formulation. In yet another example, the solvent system comprises Forskolin in an amount of 0.055% (wt/wt) of the total formulation. In yet another example, the solvent system comprises Forskolin in an amount of 0.06% (wt/wt) of the total formulation. In yet another example, the solvent system comprises Forskolin in an amount of 0.065% (wt/wt) of the total formulation. In yet another example, the solvent system comprises Forskolin in an amount of 0.07% (wt/wt) of the total formulation. In yet another example, the solvent system comprises Forskolin in an amount of 0.075% (wt/wt) of the total formulation.

In one example, the solvent system comprises a skin stabilizer in an amount of 0.01% (wt/wt) of the total formulation. In another example, the solvent system comprises a skin stabilizer in an amount of 0.02% (wt/wt) of the total formulation. In yet another example, the solvent system comprises a skin stabilizer in an amount of 0.03% (wt/wt) of the total formulation. In yet another example, the solvent system comprises a skin stabilizer in an amount of 0.04% (wt/wt) of the total formulation. In yet another example, the solvent system comprises a skin stabilizer in an amount of 0.05% (wt/wt) of the total formulation. In yet another example, the solvent system comprises a skin stabilizer in an amount of 0.06% (wt/wt) of the total formulation. In yet another example, the solvent system comprises a skin stabilizer in an amount of 0.07% (wt/wt) of the total formulation. In yet another example, the solvent system comprises a skin stabilizer in an amount of 0.08% (wt/wt) of the total formulation. In yet another example, the solvent system comprises a skin stabilizer in an amount of 0.09% (wt/wt) of the total formulation. In yet another example, the solvent system comprises a skin stabilizer in an amount of 0.10% (wt/wt) of the total formulation. In yet another example, the solvent system comprises a skin stabilizer in an amount of 0.15% (wt/wt) of the total formulation. In yet another example, the solvent system comprises a skin stabilizer in an amount of 0.20% (wt/wt) of the total formulation. In yet another example, the solvent system comprises a skin stabilizer in an amount of 0.25% (wt/wt) of the total formulation. In yet another example, the solvent system comprises a skin stabilizer in an amount of 0.30% (wt/wt) of the total formulation. In yet another example, the solvent system comprises a skin stabilizer in an amount of 0.35% (wt/wt) of the total formulation. In yet another example, the solvent system comprises a skin stabilizer in an amount of 0.40% (wt/wt) of the total formulation. In yet another example, the solvent system comprises a skin stabilizer in an amount of 0.45% (wt/wt) of the total formulation. In yet another example, the solvent system comprises a skin stabilizer in an amount of 0.50% (wt/wt) of the total formulation. In yet another example, the solvent system comprises a skin stabilizer in an amount within a range of the amounts described in this paragraph. The skin stabilizer can be one or more selected from the group consisting of Lauricidin®, D-panthenol (Dexpanthenol), and phytantriol.

### Administration

In some examples, transdermal insulin formulations disclosed herein allow the delivery of insulin directly into the patient's body where insulin receptors in the cells are located. As such, the transdermal insulin formulation(s) described herein provide equivalent percentages of bioavailability as by injection of insulin to a subject in need thereof.

In some examples, transdermal insulin formulations disclosed herein are applied to any area of skin, such as, for example planter foot arch, lateral ankle, palm, upper arm, ventral forearm, dorsal forearm, back, chest, thigh, abdomen, groin, scalp, axilla, forehead, lower back, buttocks, among others. In these embodiments, most suitable sites to apply transdermal insulin formulations disclosed herein are ventral forearm, upper arm, and chest.

In some examples, transdermal insulin formulations disclosed herein include liquid dosage forms, such as, for example solutions, liquid sprays, lotions, and the like.

In one example, method of administering the formulation as described herein comprises using a spray device. Spray devices can be single dose or multiple dose systems, for example comprising a bottle, pump and actuator, and are available from various commercial sources. As an example, for a spray device, the typical volume of liquid that is dispensed in a single spray actuation is from 0.01 ml, 0.02 ml, 0.03 ml, 0.04 ml, 0.05 ml, or 0.06 to 0.14 ml, for example from 0.08 to 0.12 ml, such as 0.1 ml.

In some examples, transdermal insulin formulations disclosed herein can be designed for fast release and transdermal absorption of insulin. In other examples, transdermal insulin formulations disclosed herein can be designed for slow release and transdermal absorption of insulin over a prolonged period of time.

In some examples, transdermal insulin formulations disclosed herein are administered in a single administration whereby a certain amount of insulin is administered at once. In other examples, transdermal insulin formulations disclosed herein are administered by multiple administrations in one or more sub-doses over a specified period of time.

In some examples, transdermal insulin formulations disclosed herein may be tailored for individual patients according to clinical symptoms and baseline serum concentrations of blood glucose. In these embodiments, transdermal pharmaceutical compositions may be prescribed with various concentrations of insulin and suitable dosage regimens to more closely mimic the physiological pulsatile secretion of insulin, thereby keeping the serum glucose levels within physiologic range.

In an example, the transdermal insulin formulation(s) as described herein are administered within a dosage range from about 25 IU/day to about 500 IU/day of insulin.

In one example, disclosed herein is a method for delivering insulin to a subject in need thereof, the method comprising administering to the subject a therapeutically effective amount of a transdermal insulin formulation described herein.

In one example, disclosed herein is a method for stabilizing glucose levels in a subject receiving insulin, comprising administering to the subject in need thereof a therapeutically effective amount of a transdermal insulin formulation described herein.

In one example, disclosed herein is a method for treating diabetes, comprising administering to the subject in need thereof a therapeutically effective amount of a transdermal insulin formulation described herein.

In one example, a method comprising administering to a subject in need thereof a therapeutically effective amount of a transdermal insulin formulation described herein and further administering at least one additional therapeutic agent. The at least one therapeutic agent can be subcutaneous administration of another insulin and/or any other therapeutic agent available in the market for stabilizing glucose levels, stimulating native insulin production and/or otherwise treating diabetes, in a subject. For example, such additional therapeutic agents include, but are not limited to, Metformin and repaglinide.

In one example, disclosed herein is a method of delivering 90% or more of the at least one insulin rapidly through the skin to the underlying fatty tissue, Interstitium and capillary plexus. This delivery may be accomplished in only a few to several tens of seconds or just a few minutes or less.

The instant application also discloses a method for preparing a transdermal insulin formulation as described herein. In one example, the method comprises (a) selecting the at least one insulin, (b) determining an effective dose of said at least one insulin, said effective dose of said at least one insulin having molecular properties including van der Waals forces and dipole moments, (c) quantifying said molecular properties of said at least one insulin, (d) determining an amount of a solvent system to solubilize said effective dose of at least one insulin, said amount of the solvent system having molecular properties including van der Waals forces and dipole moments: (e) quantifying said molecular properties of said amount of the solvent system, (f) comparing said molecular properties of the at least one insulin and said molecular properties of the solvent system, (g) determining that the molecular properties of the solvent system without insulin are substantially the same or approximately ±20% as the molecular properties of the at least one insulin, and (h) combining said solvent system and said at least one insulin to provide the transdermal insulin formulation.

In one example, the method for preparing a transdermal insulin formulation as described herein comprises selecting one or more ingredients for the solvent system for determining an amount of a solvent system to solubilize said effective dose of at least one insulin, said one or more ingredients are selected from the group consisting of two or more solvents, a solvent modifier, a solute modifier, a source of cellular activation energy, a skin stabilizer, and a combination thereof: each of said one or more ingredients having molecular properties including van der Waals forces and dipole moments.

In another example, the method for preparing a transdermal insulin formulation as described herein comprises selecting one or more ingredients for the solvent system for determining an amount of a solvent system to solubilize said effective dose of at least one insulin, said one or more ingredients are selected from the group consisting of two or more solvents, a solvent modifier, a solute modifier, a source of cellular activation energy, a skin stabilizer, one membrane permeability modifier, at least one enzyme activator, at least one capillary dilator and a combination thereof: each of said one or more ingredients having molecular properties including van der Waals forces and dipole moments.

In one example, disclosed herein is a method of selecting the ingredients and amounts to prepare a transdermal insulin formulation as described herein, wherein the method comprises the steps of: (a) selecting at least one insulin necessary to treat a specific condition, (b) quantifying the amount of said insulin for an effective dose, (c) quantifying the molecular properties of said insulin to include van der Waals forces and the sum of mol-moments, (d) surveying solvents for said insulin, (e) quantifying the amounts of said solvents to solubilize said insulin, (f) quantifying the molecular properties of said solvents to include van der Waals forces and dipole-moments, (g) comparing the molecular properties of said solvents to said molecular properties of said insulin, (h) determining additional ingredients to form a solvent system for transmigration, (i) quantifying the molecular properties of said additional ingredients to include van der Waals forces and mol-moments, (j) determining a weighted sum of said molecular properties of said additional ingredients and said molecular properties of said solvents to determine molecular properties of said solvent system, (k) summing said molecular properties of said solvent system and said insulin: (l) comparing (j) and (k), and (m) adjusting said solvent system wherein said molecular properties of said at least one insulin in the delivery system are substantially the same as said molecular properties of said solvent system without insulin.

In one example described herein, van der Waals forces and/or dipole-moments of the at least one insulin in the delivery system are approximately ±20% of van der Waals forces and/or dipole-moments of said solvent system. In one example described herein, van der Waals forces and/or dipole-moments of the at least one insulin in the delivery system are approximately ±15% of van der Waals forces and/or dipole-moments of said solvent system. In one example described herein, van der Waals forces and/or dipole-moments of the at least one insulin in the delivery system are approximately ±10% of van der Waals forces and/or dipole-moments of said solvent system. In one example described herein, van der Waals forces and/or dipole-moments of the at least one insulin in the delivery system are approximately ±5% of van der Waals forces and/or dipole-moments of said delivery system.

The transdermal insulin formulations as described herein were developed in series and tested on a single patient described as a brittle Type-2 Diabetes (T2D) patient with low insulin sensitivity. The experiments demonstrated that the transdermal insulin formulations disclosed herein:
(a) can be supplemented transdermally on a one-to-one basis with the injectable dose form,
(b) can be delivered in fast and long-acting forms as well as Humulin with no difference in results, obviating the need for needle injectors,
(c) results in flatter insulin profiles and minimizes excursions,
(d) avoids tendency to hypoglycemia by localizing the receptor effect to the area applied when used exclusively, and
(e) temporarily enhances insulin sensitivity even for injected forms.

The following examples are intended to illustrate the scope of the disclosure and are not intended to be limiting. It is to be understood that other formulations known to those skilled in the art may alternatively be used.

### EXAMPLES

### Example 1

### Process for Preparation of Transdermal Insulin Formulation

Weigh out Solvents (Anhydrous Ethanol, Propylene glycol or Carbonate and acetone and admix in a reactor vessel at ambient temperature and with moderate stirring.

Weigh out excipients, crushing two crystalline forms, glycerol monolaurate and MSM to powder to accelerate dissolution, and admix into the reactor vessel.

Weigh out and add other liquid and semi-solid excipient ingredients and admix into the reactor vessel except for phosphoric acid.

Weigh out and add phosphoric acid and admix into the reactor vessel.

Add the appropriate weight of Insulin to the reactor vessel.

Allow to mix for approximately 1 hour until a clear solution is achieved.

The transdermal insulin formulation thus prepared was used, in the amounts indicated, in the following examples. Unless otherwise noted all the ingredients are USP grade. Insulin as referred to herein may be any form of insulin including, rapid-acting, short-acting, intermediate acting, and/or long acting.

### Example 2

### Formulations

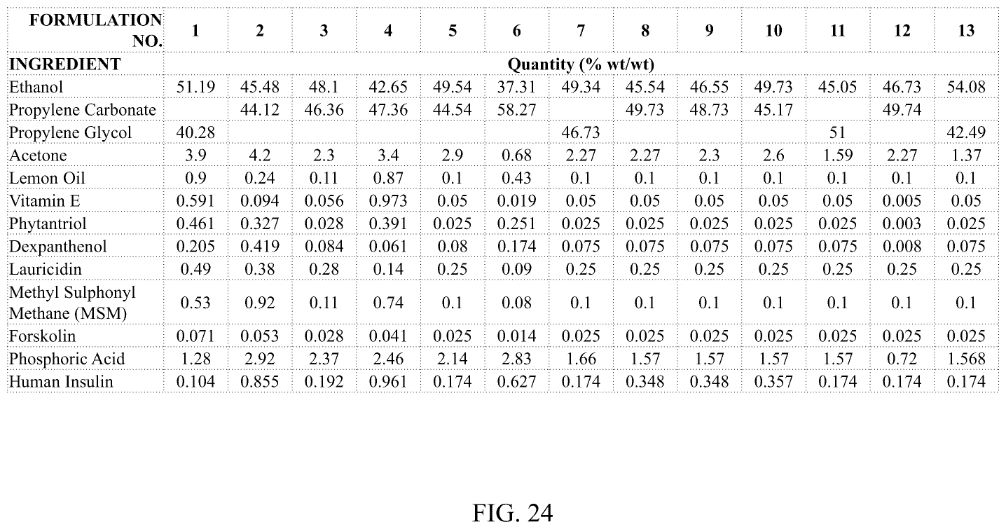

Transdermal insulin formulation were prepared using the above process comprising insulin and solvent system described herein. Examples of such formulations are highlighted in FIG. 24. The range of amounts considered for each ingredient of the formulation is described in Table 2 below:

TABLE 2

| | Amount range (wt/wt of the |
|---|---|
| Ingredient | total formulation) |
| Ethanol | 35%-50% |
| Propylene Carbonate or Propylene Glycol | 40%-55% |
| Acetone | 0.5%-5.0% |
| Lemon oil | 0.001%-1.0% |
| Vitamin E | 0.001%-1.0% |
| Phytantriol | 0.01%-0.50% |
| D-panthenol | 0.01%-0.50% |
| Lauricidin ® | 0.01%-0.50% |
| Methylsulfonylmethane (MSM) | 0.001%-1.0% |
| Forskolin | 0.001%-0.075% |
| Phosphoric acid | 0.25%-3.0% |
| Insulin | 0.1%-6% |

The insulin potency of the resulting formulations ranged from 10 IU/ml to 1600 IU/ml. Three formulations with an insulin potency of 200 IU/mL were selected for clinical testing.

### Example 3

### Clinical Testing

The formulations described in Example 2 were used in clinical testing of a single patient. The basic clinical protocol is as following:
(a) A single dose of Novolog® was administered SC at around 6 a.m. to titrate sugar to a targeted 9 a.m. level, somewhere around 100 mg/dL,
(b) With one exception, Lantus® (long-acting basal insulin) was used as background support. Upon identifying a sharp drop in Lantus® effect around 2 PM, Lantus dose was bifurcated to BID, whereupon reduced post prandial spikes and leveling continued,
(c) First transdermal dose was administered at approximately 9 AM each day,
(d) Transdermal Insulin was sprayed onto the inner aspect of the forearm or the chest by means of a metered (0.2 mL/pump) finger-actuated sprayer and rubbed in, and
(e) Blood glucose levels were tested at baseline pre-dose then hourly thereafter.

### Experiment 1:

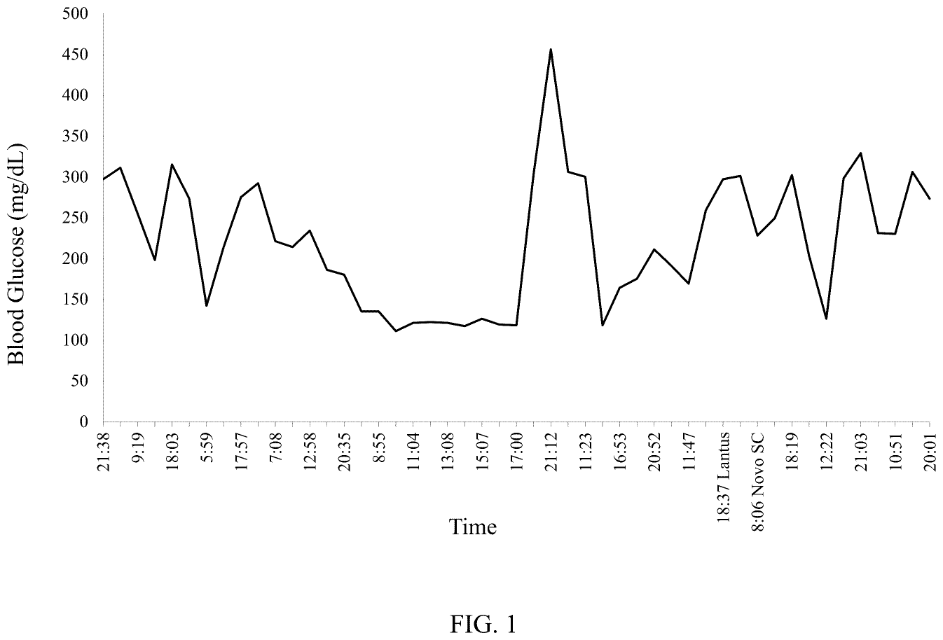

The formulation delivered insulin at 209 IU/mL to evaluate performance. Blood glucose was monitored three days prior and post. Patient fasted for 24 hours, Transdermal (TD) insulin was administered 15 hours post prandial, approximately every 3-4 hours. Blood glucose held at approximately 120 mg/dL until one hour post prandial (10 hours post transdermal administration). Blood glucose began rising as TD administration was reduced and returned to pre-TD values 12 hours post transdermal administration. As shown in FIG. 1, and Table 3 below. Insulin sensitivity with post transdermal administration values were higher than pre dosing through 50 hours (last measurement taken).

TABLE 3

| | Blood | | Blood | Expected | Sensitivity |
|---|---|---|---|---|---|
| | Glucose | | Glucose | Blood | Expected/ |
| Time | mg/dL | Dose | Adj. | Glucose | Found |
| 21:38 | 297 | 80 | | | |
| Novolog ® SC | | | | | |
| 5:59 | 311 | 80 | −56 | 353 | 2.27 |
| 9:19 | 255 | 80 | | | |
| 12:53 | 198 | 80 | | | |
| 18:03 | 315 | 80 | | | |
| 21:29 | 273 | 80 | | | |
| 5:59 | 142 | 40 | −56 | 329 | 4.63 |
| 9:32 | 214 | 80 | | | |
| 15:49 | | 80 | | | |
| 17:57 | 275 | 90 | | | |
| 21:19 | 292 | 80 | | | |
| 7:08 | 221 | 80 | −56 | 348 | 3.15 |
| 9:32 | 214 | 80 | | | |
| 12:58 | 234 | 80 | | | |
| 19:12 | 186 | 80 | | | |
| 20:35 | 180 | 40 | | | |
| 6:38 | 135 | 40 | −190 | 370 | 5.48 |
| Novolog ® & | | | | | |
| Lantus ® SC | | | | | |
| 8:55 TD | 135 | 80 | 22 | 113 | 1.67 |
| 10:04 | 111 | | | | |
| 11:04 | 121 | 80 | −15 | 126 | 2.09 |
| 12:01 | 122 | | | | |
| 13:08 | 121 | 80 | 15 | 107 | 1.76 |
| 14:01 | 117 | | | | |
| 15:07 | 126 | 80 | 39 | 78 | 1.23 |
| 16:01 | 119 | | | | |
| 17:00 | 118 | 121 | 46 | 73 | 1.23 |
| 19:37 | 303 | | | | |
| Lantus ® | | | | | |
| 21:12 | 456 | 121 | 17 | 286 | 1.25 |
| 8:03 TD | 306 | 121 | −55 | 511 | 3.34 |
| Novolog ® & | | | | | |
| Lantus ® | | | | | |
| 11:23 TD | 300 | 121 | −20 | 326 | 2.17 |
| 11:23 | | 100 | | | |
| Novolog ® SC | | | | | |
| 14:21 | 118 | 80 | 180 | 120 | 2.03 |
| 15:07 | | | | | |
| 16:53 | 164 | 80 | −31 | 148 | 1.81 |
| 19:08 | 175 | 80 | 4 | 160 | 1.83 |
| Lantus ® SC | | | | | |
| 19:08 | | 40 | | | |
| Novolog ® SC | | | | | |
| 20:52 TD | 211 | 80 | 46 | 129 | 1.22 |
| 20:52 | | 60 | | | |
| Novolog ® SC | | | | | |
| 8:23 TD & | 191 | 121 | −17 | 228 | 2.39 |
| Lantus ® SC | | | | | |
| 8:23 | | 70 | | | |
| Novolog ® SC | | | | | |
| 11:47 TD | 169 | 121 | 135 | 55 | 0.65 |
| 11:47 | | 50 | | | |
| Novolog ® SC | | | | | |
| 14:34 | 259 | 121 | 98 | 71 | 0.54 |
| 18:37 TD & | 297 | 121 | 6 | 252 | 1.70 |
| Lantus ® SC | | | | | |
| 18:37 | | 100 | | | |
| Novolog ® SC | | | | | |
| 21:40 | 301 | 60 | 166 | 131 | 0.87 |
| Last TD | | | | | |
| Novo | | 100 | | | |
| 8:06 | 228 | 80 | 50 | 251 | 2.20 |
| Novolog ® & | | | | | |
| Lantus ® SC | | | | | |
| 13:07 | 249 | 80 | | | |
| 18:19 | 302 | 150 | | | |
| Novolog ® & | | | | | |
| Lantus ® SC | | | | | |
| 0:30 | 203 | 50 | | | |
| 7:45 | | 80 | | | |
| Novolog ® & | | | | | |
| Lantus ® SC | | | | | |
| 12:22 | 126 | 40 | | | |
| 18:38 | 298 | 100 | | | |
| Novolog ® & | | | | | |
| Lantus ® SC | | | | | |
| 21:03 | 329 | 100 | | | |
| 6:02 | 231 | 80 | −16 | 345 | 2.99 |
| Novolog ® & | | | | | |
| Lantus ® SC | | | | | |
| 10:51 | 230 | 80 | | | |
| 17:49 | 306 | 80 | | | |
| Novolog ® & | | | | | |
| Lantus ® SC | | | | | |
| 20:01 | 273 | 90 | | | |

| | |
|---|---|
| Select Average | 1.85 |

### Experiment 2:

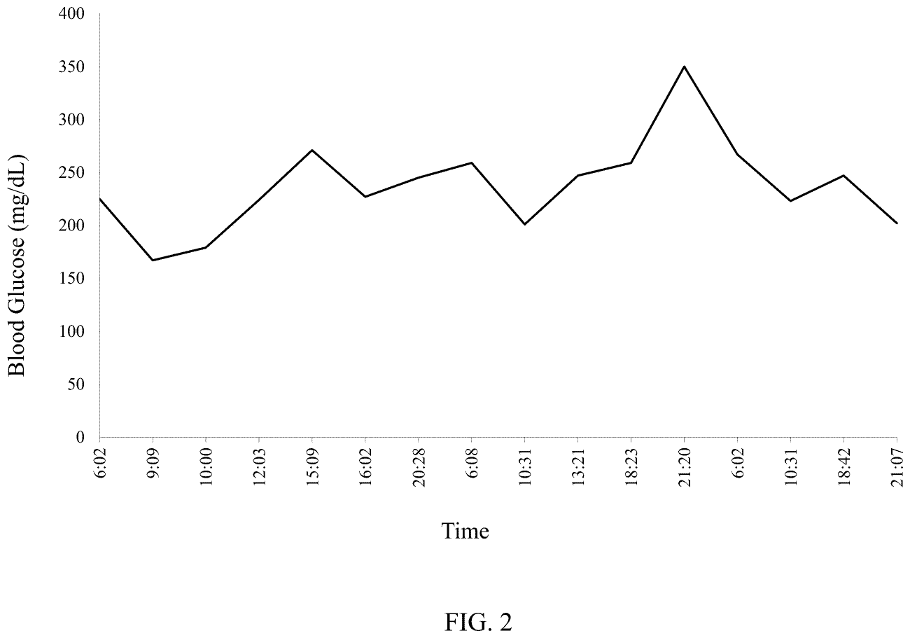

The formulation delivered insulin at 201 IU/mL to evaluate performance. Blood glucose was again monitored three days prior and post. Transdermal insulin was administered 15 hours post prandial at large bolus (201 IU) approximately every 3-8 hours. Blood glucose was held within approximately ±20 mg/dL of the pre transdermal value through day 3, where lunch was omitted (12 hour fast). Blood glucose began declining as transdermal administration was reduced with increased Novolog® administration and remained lower than pre transdermal for 72 hours. As shown in FIG. 2, and Table 4 below, Insulin sensitivity with post transdermal administration values were higher than pre dosing through 50 hours (last measurement taken).

TABLE 4

| | Blood | | Blood | Expected | Sensitivity |
|---|---|---|---|---|---|
| | Glucose | | Glucose | Blood | Expected/ |
| Time | mg/dL | Dose | Adj. | Glucose | Found |
| 6:02 | 225 | 90 | −90 | 363 | 3.23 |
| Novolog ® & | | | | | |
| Lantus ® SC | | | | | |
| 9:09 TD | 167 | 201 | 90 | 135 | 1.62 |
| 10:00 | 179 | | | | |
| 12:03 | 224 | 201 | −34 | 213 | 1.9 |
| 15:09 | 271 | 201 | 87 | 137 | 1.01 |
| 16:02 | 227 | 90 | | | |
| Novolog ® SC | | | | | |
| 18:43 | | 201 | | | |
| 20:28 | 245 | 90 | | | |
| Novolog ® & | | | | | |
| Lantus ® SC | | | | | |
| 6:08 | 259 | 100 | −90 | 335 | 2.59 |
| Novolog ® & | | | | | |
| Lantus ® SC | | | | | |
| 6:08 TD | | 201 | | | |
| 10:31 | 201 | 201 | | | |
| 13:21 | 247 | 201 | 64 | 137 | 1.11 |
| 18:23 TD & | 259 | 201 | 167 | 80 | 0.62 |
| Lantus ® SC | | | | | |
| 21:20 | 350 | 100 | 44 | −44 | −0.25 |
| 6:02 TD | 267 | 201 | −43 | 393 | 2.94 |
| 6:02 | | 100 | | | |
| Novolog ® & | | | | | |
| Lantus ® SC | | | | | |
| 10:31 No | 223 | 201 | 273 | −6 | −0.05 |
| Lunch | | | | | |
| 18:42 | 247 | 201 | 162 | 61 | 0.49 |
| TD & | | | | | |
| Lantus ® SC | | | | | |
| 21:07 | 202 | 40 | 342 | −95 | −0.94 |
| Last TD | | | | | |
| 21:07 | | 50 | | | |
| Novolog ® SC | | | | | |
| 6:00 | 193 | 80 | −163 | 365 | 3.78 |
| Novolog ® & | | | | | |
| Lantus ® SC | | | | | |
| 12:24 | 185 | 80 | | | |
| 17:56 | 127 | 80 | | | |
| Novolog ® & | | | | | |
| Lantus ® SC | | | | | |
| 21:10 | 120 | | | | |
| 8:06 | 152 | 60 | −137 | 257 | 3.38 |
| Novolog ® & | | | | | |
| Lantus ® SC | | | | | |
| 12:13 | 194 | 80 | | | |
| 19:32 | 280 | 100 | | | |
| Novolog ® & | | | | | |
| Lantus ® SC | | | | | |
| 21:44 | 257 | 100 | | | |
| 10:06 | 207 | 80 | −124 | 381 | 3.68 |
| Novolog ® & | | | | | |
| Lantus ® SC | | | | | |
| 17:59 | 143 | 80 | | | |
| Novolog ® & | | | | | |
| Lantus ® SC | | | | | |
| 20:26 | 210 | 80 | | | |

| | |
|---|---|
| Select Average | 1.86 |

### Experiment 3:

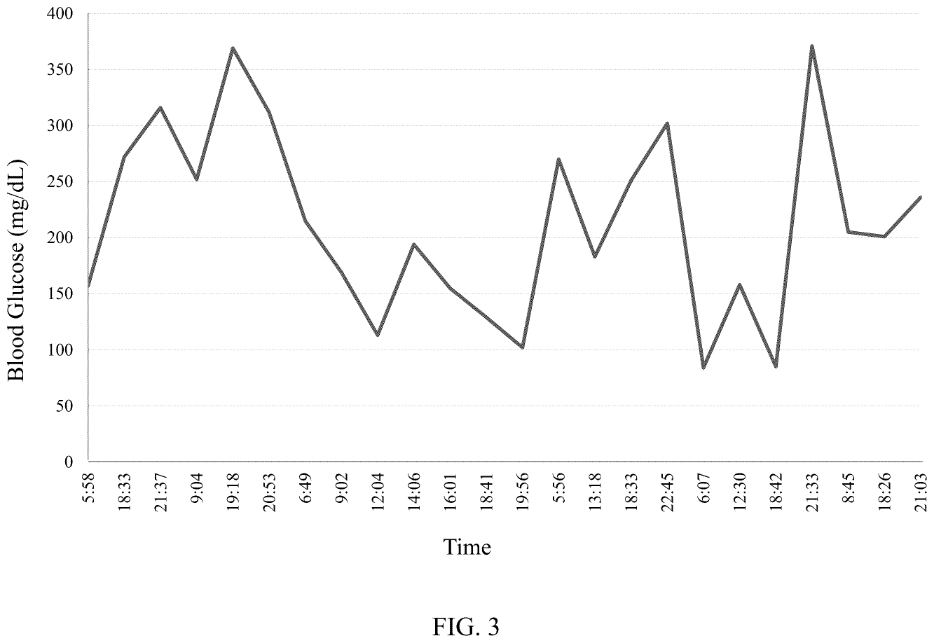

The formulation delivered insulin at 209 IU/mL to evaluate performance. Blood glucose was monitored three days prior and post. Transdermal insulin was initially administered at 9:02, 14 hours post prandial, employing a large bolus (209 IU) approximately every 2-6 hours. Novolog® was co-administered to adjust blood glucose throughout transdermal administration. As shown in FIG. 3, and Table 5 below, Insulin sensitivity with post transdermal administration values were higher than pre dosing through 50 hours (last measurement taken).

TABLE 5

| | Blood | | Blood | Expected | Sensitivity |
|---|---|---|---|---|---|
| | Glucose | | Glucose | Blood | Expected/ |
| Time | mg/dL | Dose | Adj. | Glucose | Found |
| 5:58 | 157 | 60 | −83 | 293 | 3.73 |
| Novolog ® & | | | | | |
| Lantus ® SC | | | | | |
| 12:03 | | 80 | | | |
| 18:33 | 272 | 90 | | | |
| Novolog ® & | | | | | |
| Lantus ® SC | | | | | |
| 21:37 | 316 | | 90 | 182 | 1.34 |
| 9:04 | 252 | 100 | −137 | 453 | 3.6 |
| Novolog ® & | | | | | |
| Lantus ® SC | | | | | |
| 19:18 | 369 | 140 | 10 | 242 | 1.31 |
| Novolog ® & | | | | | |
| Lantus ® SC | | | | | |
| 20:53 | 312 | 100 | 226 | 143 | 0.92 |
| 6:49 | 215 | 100 | −70 | 382 | 3.55 |
| Novolog ® & | | | | | |
| Lantus ® SC | | | | | |
| 9:02 | 169 | 40 | 146 | 69 | 0.82 |
| 9:54 TD | | 209 | | | |
| 12:04 | 113 | 209 | 34 | 135 | 2.39 |
| 14:06 | 194 | 209 | −1 | 114 | 1.18 |
| 14:06 | | 40 | | | |
| Novolog ® SC | | | | | |
| 16:01 | 155 | 40 | 37 | 157 | 2.03 |
| Novolog ® SC | | | | | |
| 18:41 | 129 | 125 | 56 | 99 | 1.54 |
| Last TD & | | | | | |
| Lantus ® SC | | | | | |
| 19:56 | 102 | 40 | 80 | 49 | 0.96 |
| 5:56 | 270 | 90 | −190 | 292 | 2.16 |
| Novolog ® & | | | | | |
| Lantus ® SC | | | | | |
| 13:18 | 183 | 60 | | | |
| 18:33 | 251 | | | | |
| 17:56 | | 80 | | | |
| Lantus ® SC | | | | | |
| 22:45 | 302 | 100 | | | |
| 6:07 | 84 | 20 | 11 | 291 | 6.93 |
| Novolog ® & | | | | | |
| Lantus ® SC | | | | | |
| 12:30 | 158 | 80 | | | |
| 18:42 | 85 | 100 | | | |
| 19:32 | | 80 | | | |
| Lantus ® SC | | | | | |
| 21:33 | 371 | 100 | | | |
| 8:45 | 205 | 80 | −97 | 468 | 4.57 |
| Novolog ® & | | | | | |
| Lantus ® SC | | | | | |
| 18:26 | 201 | 70 | | | |
| Novolog ® & | | | | | |
| Lantus ® SC | | | | | |
| 21:03 | 236 | 80 | | | |

| | |
|---|---|
| Select Average | 1.77 |

### Data Analysis:

Novolog® and Normal Insulin (Humulin®) are metabolized in 4 and 8 hours, with peak availability of 2 and 4 hours, respectively. The insulin delivery curves resemble a normal distribution, so that using ±2σ AUC gives a reasonable prediction of insulin availability.

### Expected Blood Glucose:

Expected hourly glucose rise predictions were calculated by summing: Lantus® contribution (Table 6 below), hourly contribution from Novolog® and human insulin, using the default insulin sensitivity of 2 mg/dL/IU, and comparing to the previous hour's observed blood glucose.

TABLE 6
Expected Blood Glucose (Lantus ® Sensitivity)

| | | | |
|---|---|---|---|
| Lantus ® | Net Effect/ | Lantus ® | Normal |
| Dose/Hours | Hour | Effect | Rise/Hour |

Original PD

| | | | |
|---|---|---|---|
| 110/12 | −20 | 20 | 40 |

Modified BID

| | | | |
|---|---|---|---|
| 80/12 | −27 | 13 | 40 |
| 60/12 | −30 | 10 | 40 |

### Insulin Sensitivity

Insulin sensitivity was estimated throughout by comparing the expected blood values to actual blood glucose values measured at that time point, e.g. T1=200 mg/dL, 80 IU Novolog® SC×2 mg/dL/IU=Δ−160 mg/dL, +27 mg/dL/Hr.×4 Hrs.=Δ108 mg/dL, predicted blood glucose=148.

Thus, found sensitivity values are composites of Novolog® and Human insulin, relative to the default insulin sensitivity. Values greater than 2 indicated greater-than-typical insulin sensitivity. For the SC and TD delivery, sensitivity is tabulated in the individual experiments respectively, discussed in experimental results below.

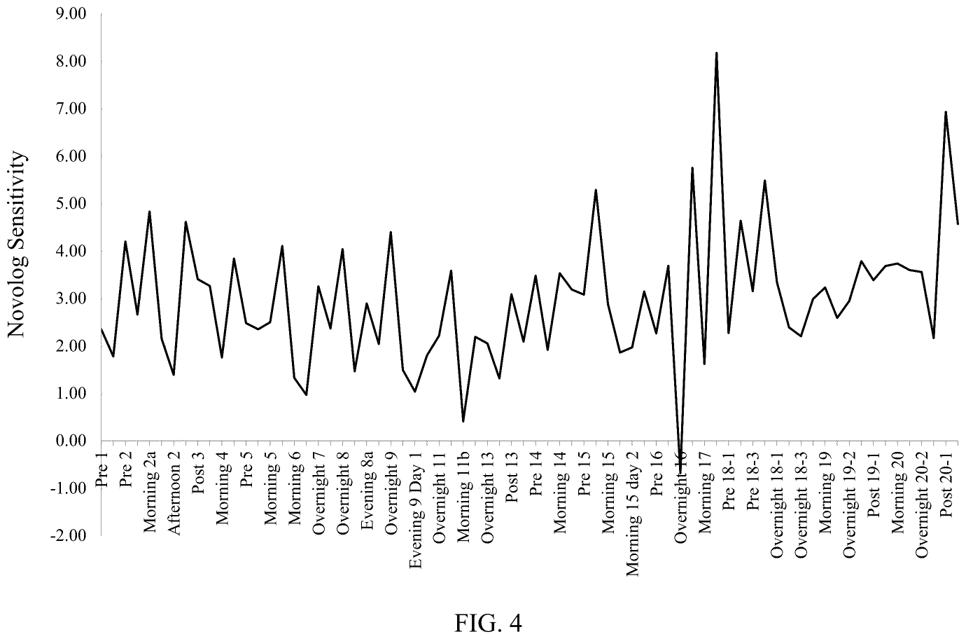

Since Novolog® SC was typically administered to bring pre-breakfast values to the desired target range (90-120 mg/dL), sensitivity was calculated in the morning and during the experiment, where possible. Novolog® sensitivity values are tabulated in Table 7 and depicted in FIG. 4. Overnight Novolog® SC sensitivity was also calculated (no food or insulin) and calculated for the days after TD dosing. This overnight sensitivity calculation included the Lantus® contribution (Table 6 above) as the only correction factor and therefore is as close to a “stand alone” estimate (no prandial correction needed) as is possible under these experimental conditions.

TABLE 7
Novolog ® Sensitivity

| | Novolog ® Sensitivity found |
|---|---|
| Pre 1 | 2.34 |
| Morning 1 | 1.77 |
| Overnight 2 | 2.66 |
| Morning 2a | 4.83 |
| Morning 2b | 2.15 |
| Afternoon 2 | 1.39 |
| Overnight 3 | 4.61 |
| Post 3 | 3.40 |
| Overnight 4 | 3.26 |
| Morning 4 | 1.75 |
| Post 4 | 3.84 |
| Pre 5 | 2.48 |
| Overnight 5 | 2.35 |
| Morning 5 | 2.50 |
| Overnight 6 | 4.10 |
| Morning 6 | 1.32 |
| Evening 6 | 0.96 |
| Overnight 7 | 3.25 |
| Morning 7 | 2.36 |
| Overnight 8 | 4.04 |
| Morning 8 | 1.46 |
| Evening 8a | 2.89 |
| Evening 8b | 2.04 |
| Overnight 9 | 4.39 |
| Morning 9 | 1.49 |
| Evening 9 Day 1 | 1.03 |
| Evening 9 Day 2 | 1.80 |
| Overnight 11 | 2.21 |
| Morning 11a | 3.58 |
| Morning 11b | 0.41 |
| Overnight 12 | 2.19 |
| Overnight 13 | 2.05 |
| Morning day 2 | 1.31 |
| Post 13 | 3.08 |
| Post 13 + 1 | 2.09 |
| Pre 14 | 3.48 |
| Overnight 14 | 1.91 |
| Morning 14 | 3.53 |
| Post 14 | 3.19 |
| Pre 15 | 3.08 |
| Overnight 15 | 5.28 |
| Morning 15 | 2.87 |
| Overnight 15 | 1.86 |
| Morning 15 Day 2 | 1.96 |
| Post 15 | 3.14 |
| Pre 16 | 2.26 |
| Morning 16 | 3.68 |
| Overnight 17 | 5.75 |
| Morning 17 | 1.62 |
| Post 17 | 8.17 |
| Pre 18-1 | 2.27 |
| Pre 18-1 | 4.63 |
| Pre 18-2 | 3.15 |
| Pre 18-3 | 5.48 |
| Morning 18 | 3.34 |
| Overnight 18-1 | 2.39 |
| Overnight 18-2 | 2.20 |
| Overnight 18-3 | 2.99 |
| Post 18 | 3.23 |
| Morning 19 | 2.59 |
| Overnight 19-1 | 2.94 |
| Overnight 19-2 | 3.78 |
| Overnight 19-3 | 3.38 |
| Post 19-1 | 3.68 |
| Post 19-2 | 3.73 |
| Morning 20 | 3.60 |
| Overnight 20-1 | 3.55 |
| Overnight 20-2 | 2.16 |
| Overnight 20-3 | 6.93 |
| Post 20-1 | 4.57 |
| Post 20-2 | 2.96 |

The insulin sensitivity calculation was made with a conservative bias for transdermal doses throughout the experiments. A “Selected Average” was reported which excluded those points where the prediction model began to deviate significantly from actual values, e.g., after repeated sequential hourly large doses, large post-prandial spikes. Based on this modeling, reduced insulin sensitivity reflects increased insulin presence in the system.

In summary, the inventors modeled normal delivery curves for both the Novolog® SC dose and the transdermal products.

### Results:

Experiment 1 was dosed in pulses of 40 to 80 IUs while the subject fasted. The last meal was at approximately 7 pm the day before transdermal dosing, which was initiated at 8:55 am. Blood glucose, which should have been increasing at an accelerated pace due to fasting, remained flat for the entire day.

Experiment 2 was dosed at boluses of 200 IUs repeatedly. Performance was as expected with leveling extending and marked increased insulin sensitivity post transdermal dosing.

Experiment 3 was a reverse paradigm where the effect of transdermal dosing coupled with subcutaneous dosing was examined. Human Insulin transdermal was dosed at boluses of 200 IUs repeatedly, with Novolog® supplementing throughout the day. Novolog® doses were substantially less than required in the absence of transdermal formulation to obtain desired blood glucose levels. Blood glucose dropped to 113 within two hours of transdermal administration and ranged below pre-transdermal levels through 72 hours.

Further, in Experiment 3, Novolog® was dosed at 6:49 am at the end of a 48-hour period of subcutaneous dosing of short and long-acting insulin and at 10:00 am, 12:00 μm, and 2:00 pm with transdermal dosing. Even though the effect of the Novolog® would have been about 85% exhausted at 10:45 pm, the levels set by Novolog® trended down with the introduction of transdermal insulin, maintained a low post-prandial spike then leveled back down to approximately the 6:49 am baseline validating the premise that the introduction of transdermal insulin into the skin makes available insulin stored in lipid tissues under the skin.

### Categorical Insulin Sensitivity Results:

Leveling was observed at lower formulation strengths and these values were aligned with expected transdermal 8-hour availability insulin dosing. This leveling suggests that excess transdermal insulin is deposited and available for subsequent use. Novolog® subcutaneous sensitivity was generally higher than transdermal formulation delivered insulin (average 2.1 vs. 1.8 respectively) in the morning. These overnight values averaged 3.1 in contrast to the pre-trial point of 2.3.

Insulin sensitivity is a measure of how well the body utilized the supplied insulin. Generally, this value decreases with time (subject age), and is generally treated as a long-term average. To understand the transdermal system, Novolog® sensitivity was measured before, between and during the experiments, and compared relative to the assumed sensitivity of 2. Table 7 and FIG. 4 show Novolog® sensitivity overnight and before transdermal insulin delivery, and, where available, overnight during breaks in transdermal dosing. Table 7 low points (sensitivity≤2.1) are as follows: 16 data points≤2.1 out of 48, with an average sensitivity of 3.33:
(a) Mornings, after breakfast
(b) Afternoons, after lunch
(c) Evenings, after dinner
(d) Overnight, after a 4-day hiatus in transdermal dosing
(c) Overnight, during experiment without overnight Lantus® dosing
(f) Novolog® sensitivity was therefore generally much higher than pre transdermal experiments and substantially higher overnight. As no treatment has shown the ability to increase insulin sensitivity, this effect is attributed to contribution from the deposited transdermal insulin, rather than a true shift in subject insulin sensitivity
(g) Morning Novolog® sensitivity values (post breakfast) are naturally more variable as prandial contributions are not incorporated. Values ranged from 0.41 to 4.83 with an average of 2.18, that average being consistent with pre transdermal experiment values.

### Aggregate Insulin Sensitivity

As shown in the above experiments, Novolog® and transdermal insulin yielded comparable results. An Aggregate insulin sensitivity calculation includes both subcutaneous and transdermal combined effect when subcutaneous insulin was incorporated during the transdermal experiments.

### Example 4

Analysis of Permeation of Products Through Artificial Skin Model into the Supporting Medium

### Overview of Artificial Skin Model Preparation:

Primary adult human dermal fibroblasts were embedded into a fibrin matrix to produce dermal equivalents (DEs). The DEs were cultured to allow the fibroblasts to remodel the matrix. Primary neonatal human keratinocytes were applied to the DE surface and cultured under liquid for 48 hours. Artificial Skin Model was cultured at the air liquid interface (ALI) until a stratified epidermis was formed. Incubation conditions for all cultures was 37±2° C. in 5±1% (v/v) CO2 at ≥95% Relative Humidity (RH).

### Permeation Study on 100 IUs/Ml Transdermal Formulation Versus Injectable Insulin on Artificial Skin Model Constructs:

Shallow well plates with Artificial Skin Model were prepared with ImL of pre-warmed fresh maintenance medium in each well. 11 μL of 100 IUs/ml of the transdermal formulation as described in the instant application, Injectable Insulin, or PBS as a blank control was applied to the Artificial Skin Model surface. Collections of the undernatent was performed at time point 0, 3, 5, 10, 15, 20, 25, 30, 40, 50 and 60 minutes.

ELISA analysis of Insulin in the undernatent was performed using a human Insulin ELISA kit (R&D systems DINS00). Back calculation of the concentration was performed using My CurveFit software).

The mean OD values for permeation of the test transdermal formulation, Injectable Insulin, or PBS control are shown in Table 8 below.

TABLE 8

| Mean OD | TIME POINT FOR ANALYSIS | | | | | | | | | | |
|---|---|---|---|---|---|---|---|---|---|---|---|
| Values | T = 0 | T = 3 | T = 5 | T = 10 | T = 15 | T = 20 | T = 25 | T = 30 | T = 40 | T = 50 | T = 60 |
| 100 IUs/ml | 0.316 | 1.005 | 0.095 | 0.169 | 0.097 | 0.238 | 0.154 | 0.233 | 0.290 | 0.422 | 0.715 |
| Transdermal | | | | | | | | | | | |
| Formulation | | | | | | | | | | | |
| Injectable | 0.345 | 0.408 | 0.076 | 0.126 | 0.094 | 0.171 | 0.128 | 0.159 | 0.261 | 0.282 | 0.306 |
| Insulin | | | | | | | | | | | |
| PBS Control | 0.273 | 0.124 | 0.117 | 0.168 | 0.118 | 0.148 | 0.225 | 0.166 | 0.287 | 0.196 | 0.152 |

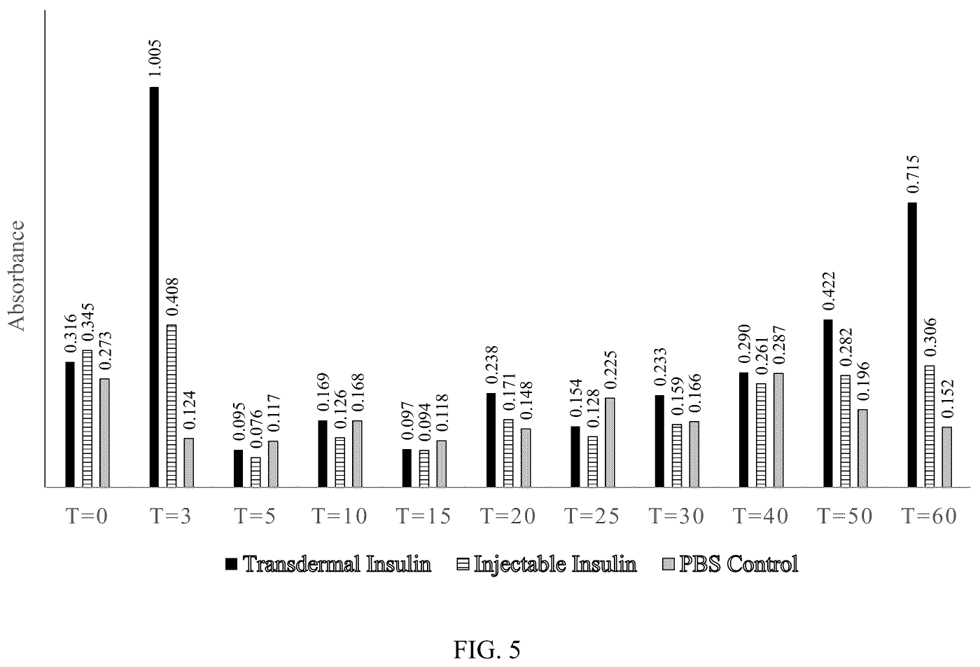

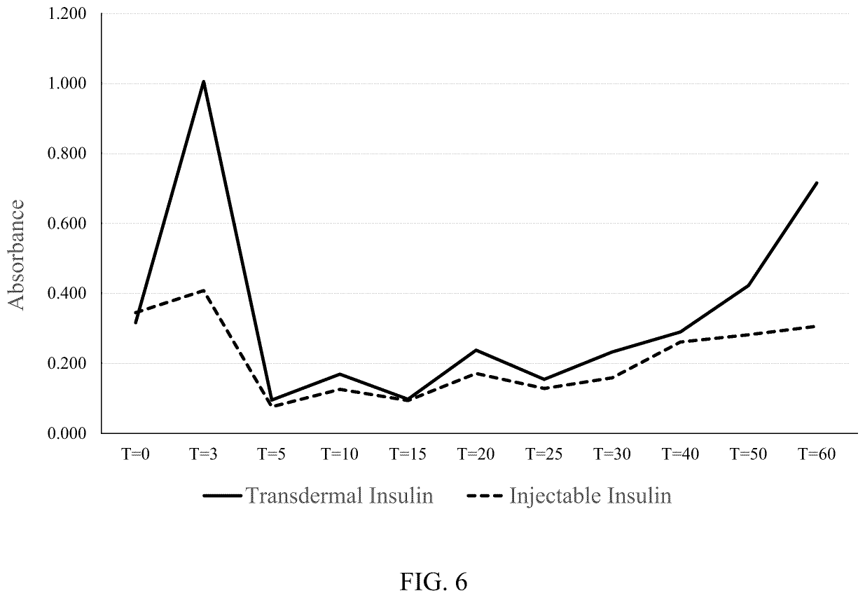

A histogram of the OD values as highlighted in Table 1 above is depicted in FIG. 5. The permeation of transdermal formulation vs. Injectable Insulin through Artificial Skin Model is presented as a graph in FIG. 6.

The back-calculated concentration of Insulin recovered in the undernatent following permeation through Artificial Skin Model is shown in Table 9 below.

TABLE 9

| Back- | | | | | | | | | | | | Total |
|---|---|---|---|---|---|---|---|---|---|---|---|---|
| calculated | TIME POINT FOR ANALYSIS | | | | | | | | | | | recovered |
| (pMol/L) | T = 0 | T = 3 | T = 5 | T = 10 | T = 15 | T = 20 | T = 25 | T = 30 | T = 40 | T = 50 | T = 60 | (pMol/L) |
| Transdermal | 101.0 | 403.0 | 8.2 | 40.3 | 8.0 | 70.2 | 33.8 | 70.0 | 93.1 | 149.9 | 276.9 | 1254.43 |
| Insulin | | | | | | | | | | | | |
| Injectable | 114.0 | 143.0 | 0.0 | 21.6 | 8.0 | 40.0 | 21.0 | 34.0 | 80.1 | 92.5 | 100.0 | 654.2 |
| Insulin | | | | | | | | | | | | |

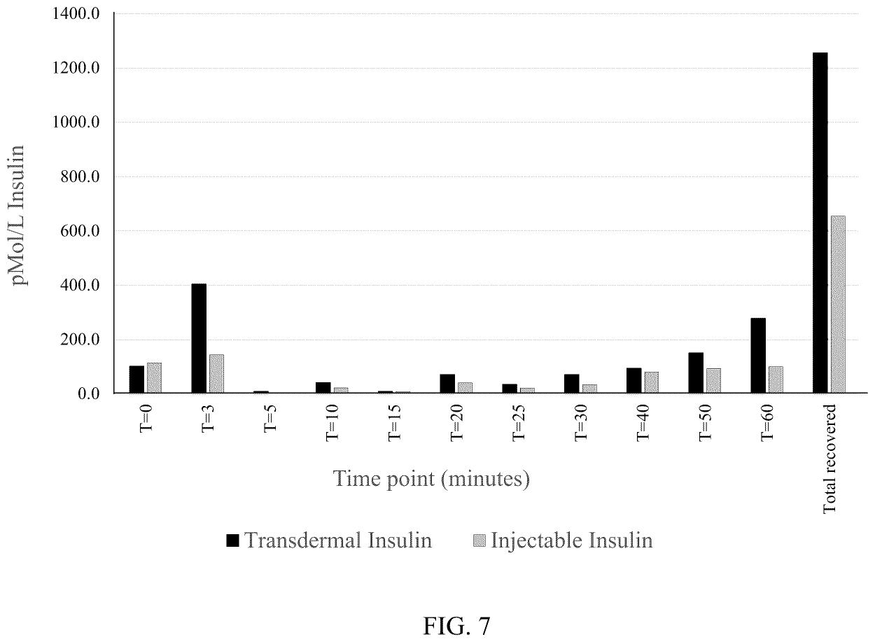

Interpolated concentrations of permeation of transdermal formulation vs Injectable Insulin through Artificial Skin Model for each timepoint, plus the total recovered amount of Insulin is presented as a graph in FIG. 7.

Controls of media only and Insulin (high, medium and low control set from Bio-Techne catalog #QC107) were also run on the ELISA plate. The standard OD values for various concentrations of Insulin, in addition to media only and the Insulin control set are shown in Table 10 below.

TABLE 10
STANDARDS

| pMol/L | OD |
|---|---|
| 500 | 1.224 |
| 250 | 0.655 |
| 125 | 0.379 |
| 62.5 | 0.224 |
| 31.25 | 0.152 |
| 15.6 | 0.102 |
| Diluent | 0.073 |
| Media | 0.067 |
| High control | 1.073 |
| Medium control | 0.526 |
| Low control | 0.228 |

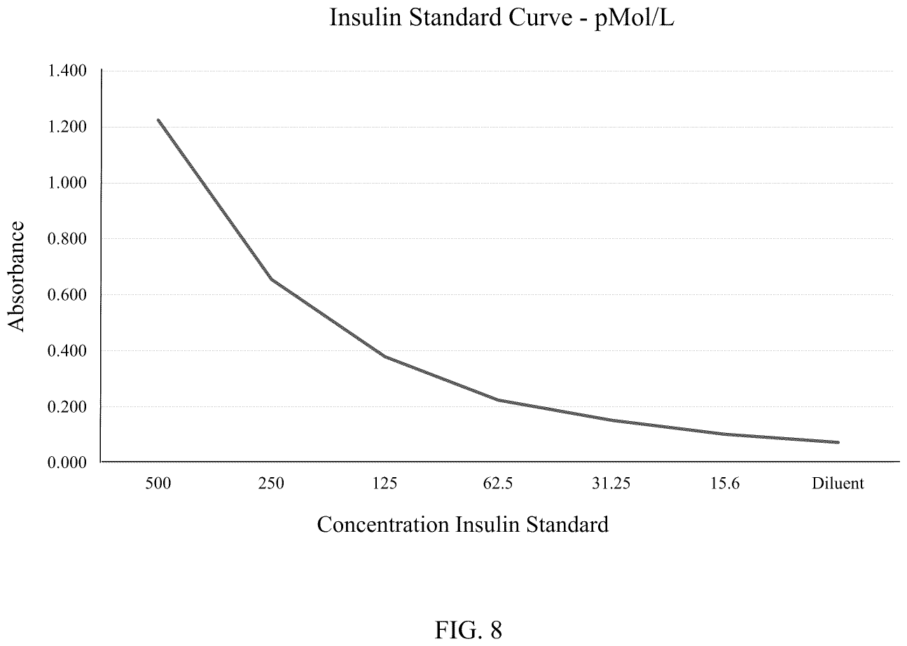

FIG. 8 shows the graph for Insulin ELISA standard curve. Using a linear plot, a R2 value of 0.9996 was obtained. The OD values for the test items was interpolated against the standard curve plot to generate pMol/L values.

The above data demonstrates that (i) the values for the transdermal formulation was higher than the Injectable Insulin at all time points, (ii) the peak concentration was noted at time point 3 minutes, with another peak at the final time point of 60 minutes, and (iii) the amount of Insulin recovered for transdermal formulation was double the amount for Injectable Insulin.

### Immunohistochemical Analysis:

Artificial Skin Model constructs were bisected and cryosectioned at 6 μm thickness at −25° C. Sections were mounted on adhesive slides, air dried and fixed in 100% ethanol before performing staining. Staining of cell structures was performed by Haematoxylin and Eosin (H&E). Visualization of insulin in the Artificial Skin Model was performed by using an anti-insulin antibody (mouse monoclonal antibody raised against human insulin, Abcam 133289). Sections were incubated with diluted antibody at concentration of 0.016 μg/ml for one hour at room temperature. Antibody was diluted in diluent/blocker (SP-5035) from Vector laboratory (USA). An indirect system was used to amplify the signal and DAB chromogen (brown) was used as an end point (PK-8200 from Vector Laboratories). All sections were micrographed at ×200 magnification on the Leica microscope.

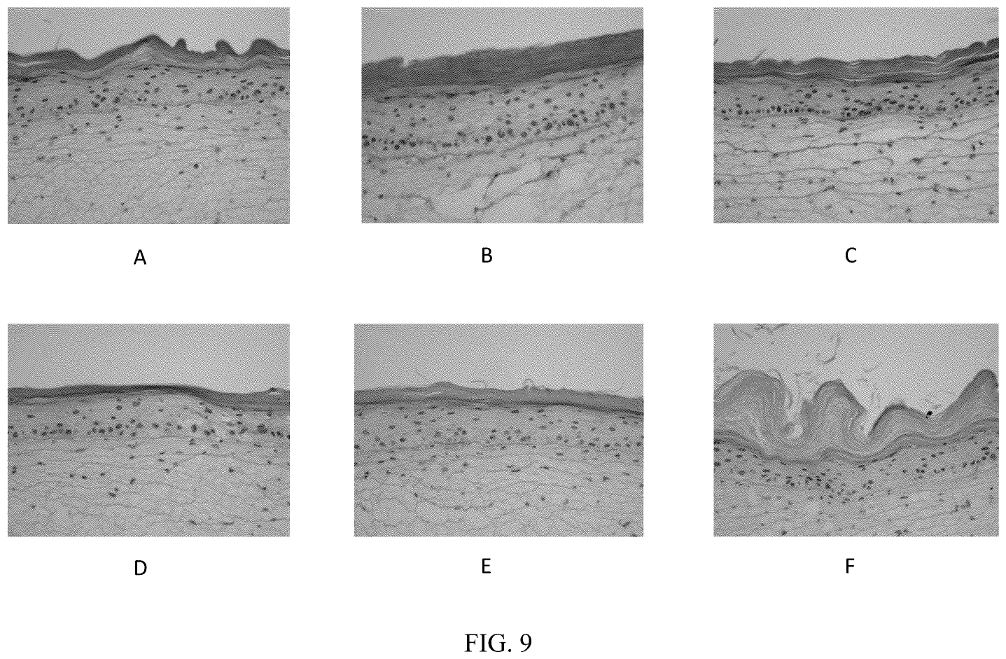

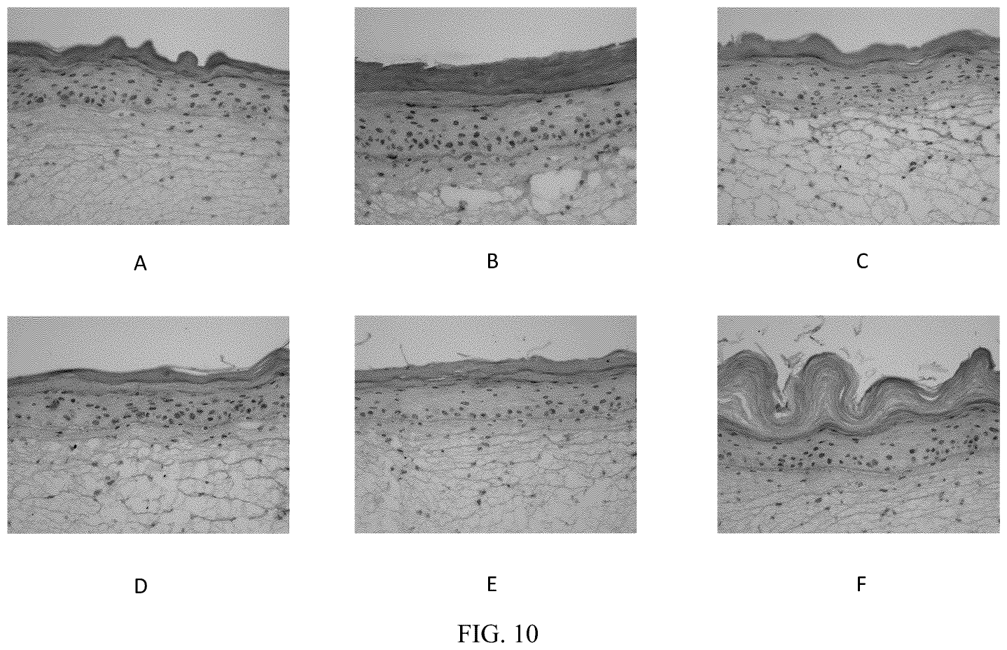

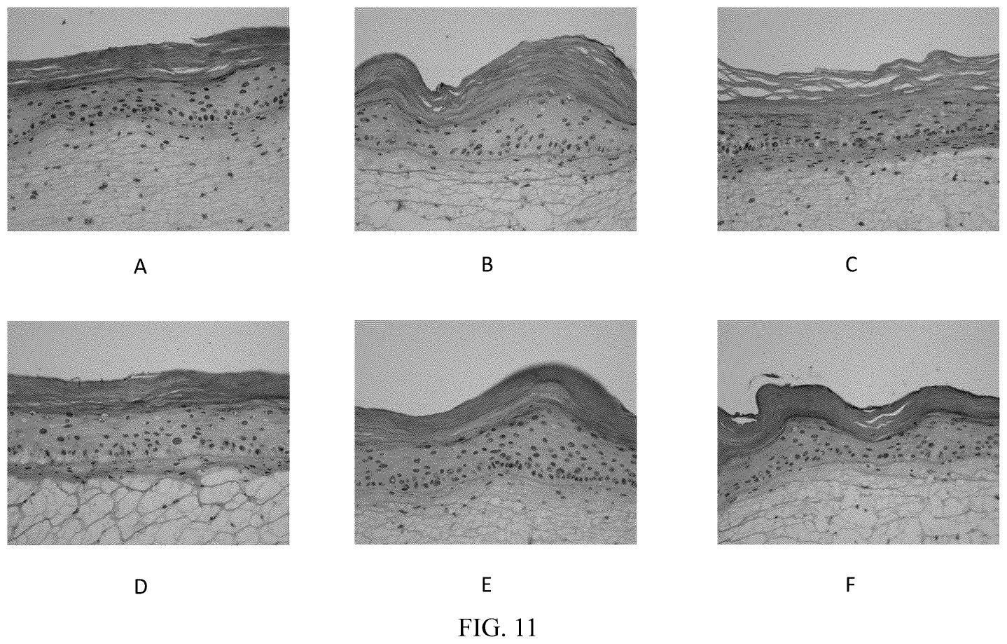

FIG. 9, FIG. 10, and FIG. 11 show staining of Artificial Skin Model constructs, wherein Fibroblast and keratinocyte cells in the constructs are stained purple and the test Insulin samples (i.e., transdermal insulin formulation and Injectable Insulin are stained brown. FIGS. 9A, 9B, 9C, 9D, 9E, and 9F show staining of control constructs at various time points, including 0 minutes, 5 minutes, 10 minutes, 20 minutes, 40 minutes, and 60 minutes, respectively. FIGS. 10A, 10B, 10C, 10D, 10E, and 10F show staining of constructs treated with injectable insulin at various time points, including 0 minutes, 5 minutes, 10 minutes, 20 minutes, 40 minutes, and 60 minutes, respectively. FIGS. 11A, 11B, 11C, 11D, 11E, and 11F show staining of constructs treated with transdermal insulin composition at various time points, including 0 minutes, 5 minutes, 10 minutes, 20 minutes, 40 minutes, and 60 minutes, respectively.

The above immunohistochemical analysis of the Artificial Skin Model constructs shows the transition of Insulin (both injectable and transdermal) from the Stratum Corneum through the epidermis and dermis.

The results obtained via permeation and immunohistochemical analyses demonstrate a faster transit with transdermal formulation as compared to Injectable Insulin.

### Example 5

### Transdermal Insulin (TD) Dose-Response Study in Control Male Rats

The following study was designed to investigate time course dose response effects of the dermal application of various transdermal formulations on fasted blood glucose in control male Wistar rats. Blood samples were also collected at the same time points after treatment to measure the levels of plasma insulin.

### Methods:

Preparation of the rat skin: On day 0 (24 hours before the first test day), a small patch (2×3 cm) of fur over the dorsum was shaved using an appropriate clipper. The area of “nude” skin was then thoroughly cleaned with cold water and dried gently patting with a dry swab. The animals were returned to housing to recover for 24 hours.

Application of the transdermal insulin: On the test day (day 1), food was removed, and animals were relocated into the clean cages with free access to tap water. After 4.5 hours, and before treatment, the basal fasted blood glucose was measured from the tail tip using Accu-Chek Glucometer, and 100 μl of Blood was withdrawn in EDTA Microvette® tubes and kept in ice before centrifugation, then plasma is collected and stored at −20 degrees C. Using a 1 m disposable plastic syringe, the insulin solutions or the vehicle were slowly applied to the prepared rat skin at 1 ml/kg of bodyweight. Simultaneously using the index finger of a gloved hand, the test material was gently massaged onto the skin. More specifically, the index finger was rotated first clockwise 10 times then counterclockwise 10 times.

Blood glucose measurement: The blood glucose was measured at 5, 10, 20, 40, 60, 90, and 120 minutes after the application of insulin solutions or vehicle, using Accu-Chek active blood glucose meter and strips.

Blood collection and plasma preparation: Blood samples were taken from the cut tip of the tail after the application of Lignocaine Gel (Biorex Laboratories UK). 100 μl of blood was collected at each time point mentioned above and treated as described above. The blood sampling was facilitated by placing animals in a warm environment (25° C.). This will not have any adverse effects on the animals. Blood was collected in EDTA-coated Microvette® tubes (Sarstedt microvette CB 300 LH, ref. 16.443, Aktiengsellschaft & Co., D-51588 Nümbrecht, Germany) for the measurement of plasma insulin concentration and stored on ice, before centrifugation at ˜500×G for 5 minutes. The resulting plasma was stored at −20 0C until required. Multiple freeze/thaw cycles were avoided.

Plasma insulin measurement: The levels of human insulin measured in the rat plasma samples were performed using the Crystal Chem human insulin ELISA kit (Catalog #90095). The Crystal Chem's Human Insulin ELISA kit is an ELISA sandwich assay for human insulin. It utilizes a specific antibody immobilized onto the microplate wells. Briefly 100 μl of human insulin antibody labelled with HRP and 25 μl of standard or plasma samples were added to each well. After two hours incubation at 37 0C, the plate was washed three times with 300 of wash buffer, then 100 μl of HRP substrate solution was added to each well and incubated in the dark at room temperature for 15 minutes. The enzymatic reaction was stopped by adding 100 μl of stop solution, and the plate was read at dual wavelengths of 450 and 630 using Spectra Max 250 (Molecular Devices. San Jose, CA 95134). The results were converted into insulin values using human insulin standards.

### Results:

The results for individual blood glucose levels are depicted in figures as described below:

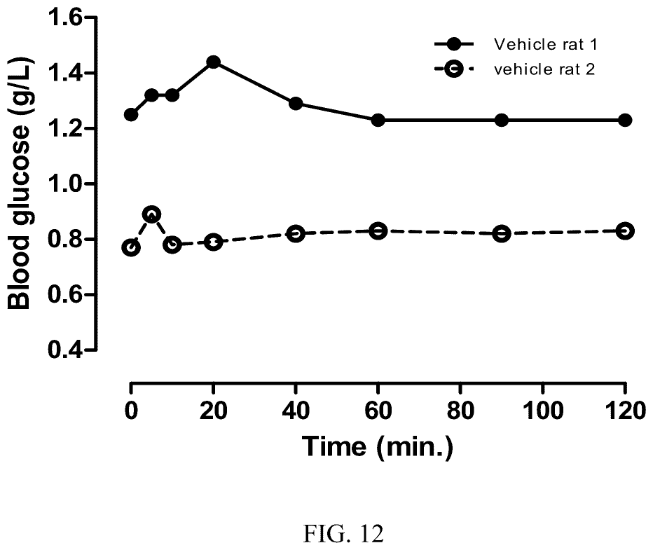

FIG. 12 shows the blood glucose levels measured following 4.5 hours fasting, using Accu-Chek®: active blood glucose meter and strips, before and after the application of 1 ml/kg of the vehicle (placebo) at 5, 10, 20, 40, 60, 90 and 120 minutes.

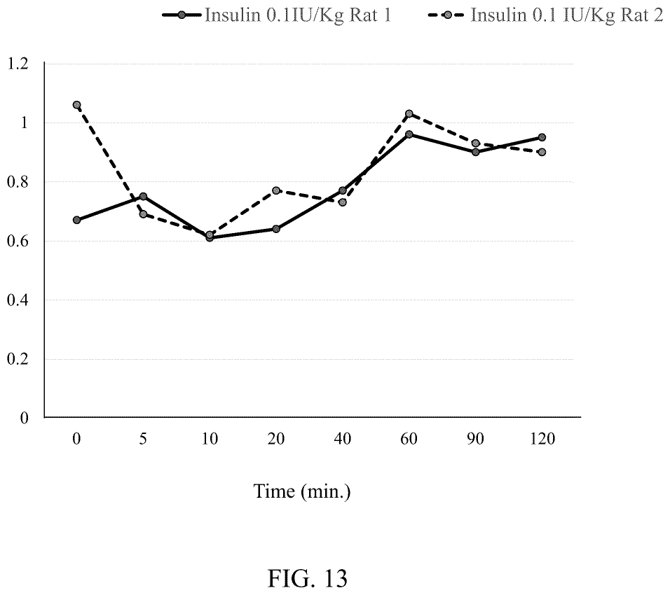

FIG. 13 shows the blood glucose levels measured following 4.5 hours fasting, using Accu-Chek®: active blood glucose meter and strips, before and after the application of human insulin solution (0.1 IU/kg/ml) at 5, 10, 20, 40, 60, 90 and 120 minutes.

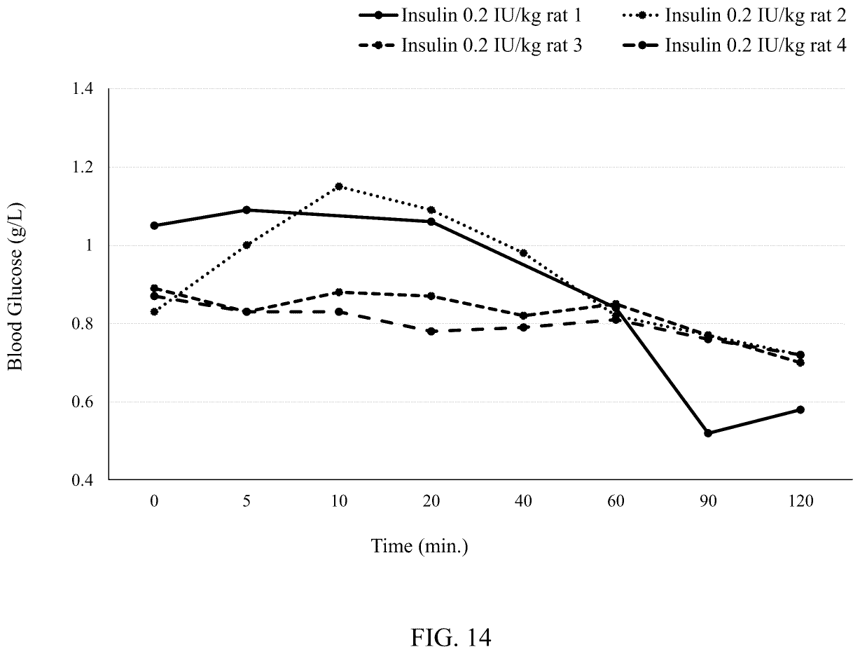

FIG. 14 shows the blood glucose levels measured following 4.5 hours fasting, using Accu-Chek® active blood glucose meter and strips, before and after the application of human insulin solution (0.2 IU/kg/ml) at 5, 10, 20, 40, 60, 90 and 120 minutes.

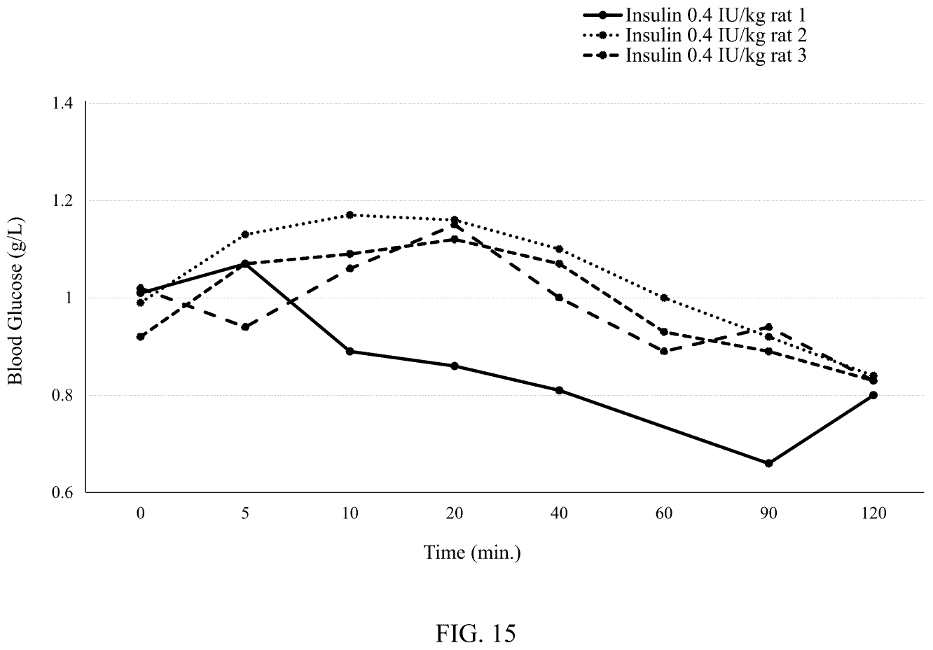

FIG. 15 shows the blood glucose levels measured following 4.5 hours fasting, using Accu-Chek® active blood glucose meter and strips, before and after the application of human insulin solution (0.4 IU/kg/ml) at 5, 10, 20, 40, 60, 90 and 120 minutes.

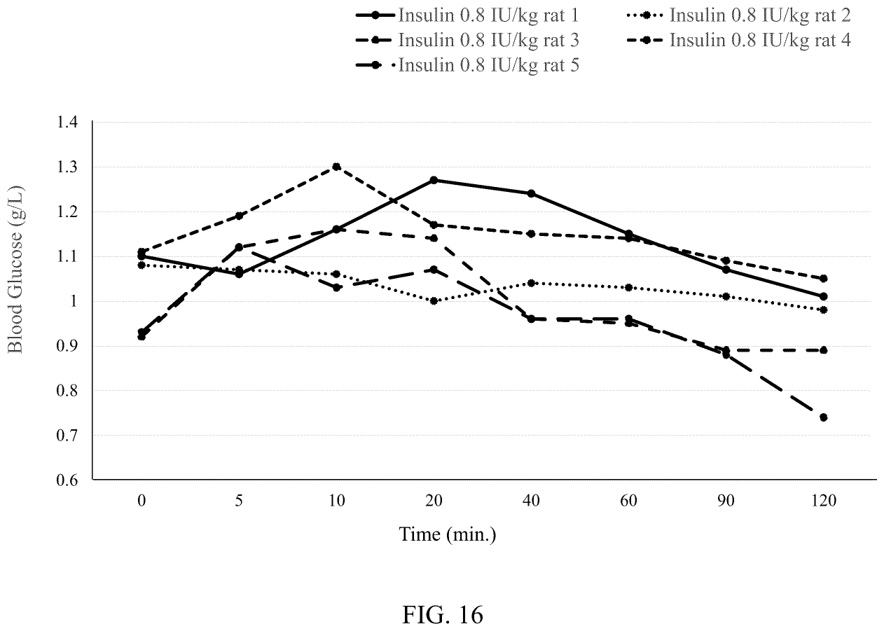

FIG. 16 shows the blood glucose levels measured following 4.5 hours fasting, using Accu-Chek® active blood glucose meter and strips, before and after the application of human insulin solution (0.8 IU/kg/ml) at 5, 10, 20, 40, 60, 90 and 120 minutes.

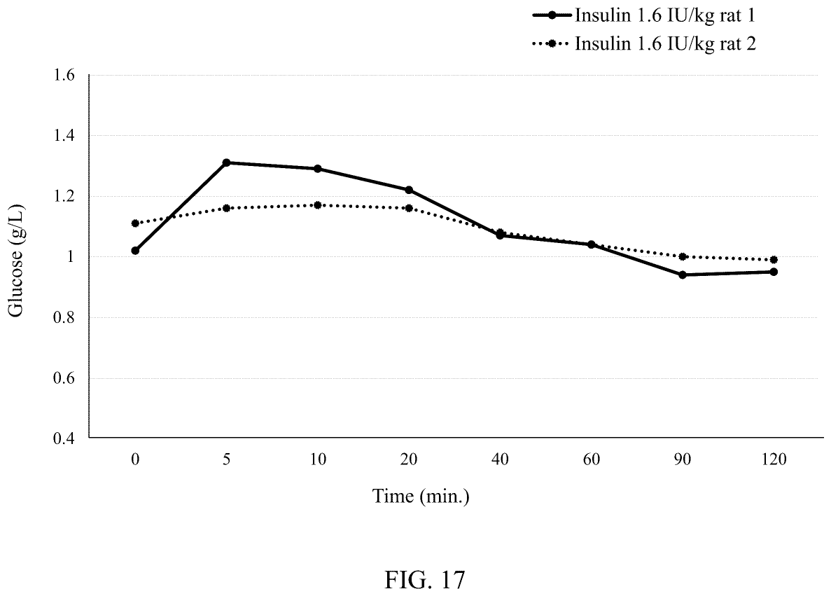

FIG. 17 shows the blood glucose levels measured following 4.5 hours fasting, using Accu-Chek® active blood glucose meter and strips, before and after the application of human insulin solution (1.6 IU/kg/ml) at 5, 10, 20, 40, 60, 90 and 120 minutes.

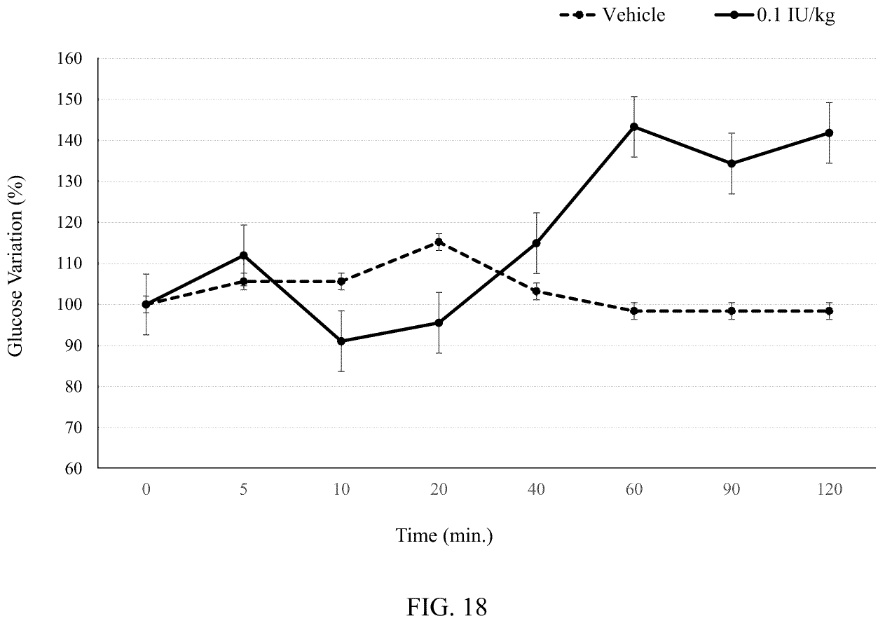

FIG. 18 shows data expressed as the percentage of variation in blood glucose levels relative to the basal levels (Time 0), after the transdermal application of human insulin solution (0.1 IU/kg/ml), or the vehicle (1 ml/kg).

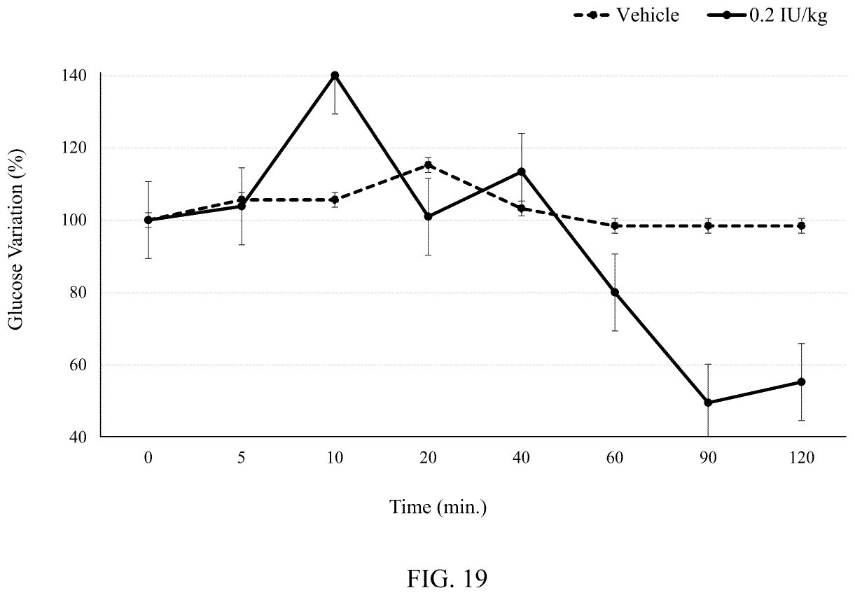

FIG. 19 shows data expressed as the percentage of variation in blood glucose levels relative to the basal levels (Time 0), after the transdermal application of human insulin solution (0.2 IU/kg/ml), or the vehicle (1 ml/kg). Statistical analysis was performed using one-way ANOVA test followed by Dunnett's multiple comparison test. Statistical significance is shown as * p<0.05.

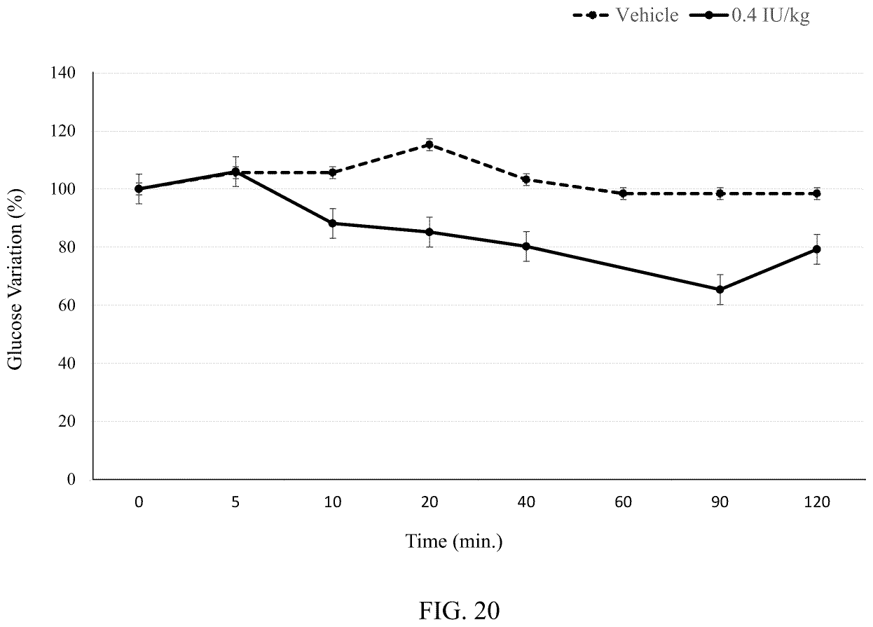

FIG. 20 shows data expressed as the percentage of variation in blood glucose levels relative to the basal levels (Time 0), after the transdermal application of human insulin solution (0.4 IU/kg/ml), or the vehicle (1 ml/kg). Statistical analysis was performed using one-way ANOVA test followed by Dunnett's multiple comparison test. Statistical significance is shown as * p<0.05, and ** p<0.01.

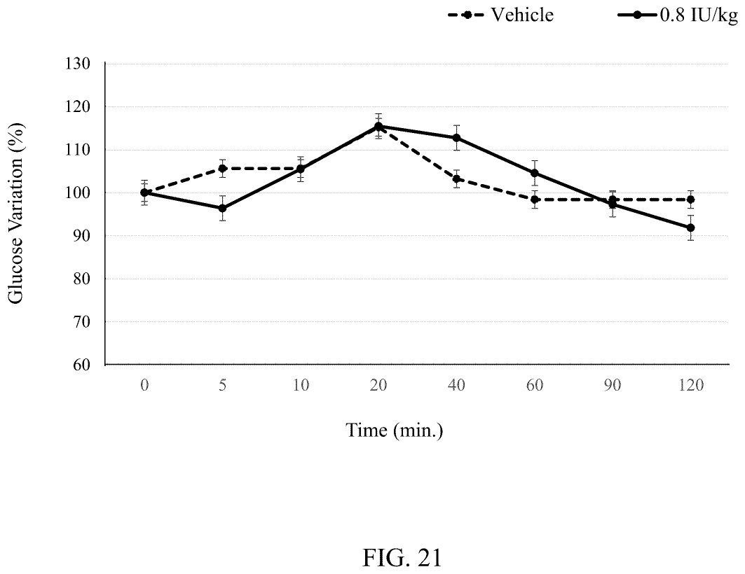

FIG. 21 shows data expressed as the percentage of variation in blood glucose levels relative to the basal levels (Time 0), after the transdermal application of human insulin solution (0.8 IU/kg/ml), or the vehicle (1 ml/kg). Statistical analysis was performed using one-way ANOVA test followed by Dunnett's multiple comparison test. Statistical significance is shown as * p<0.05.

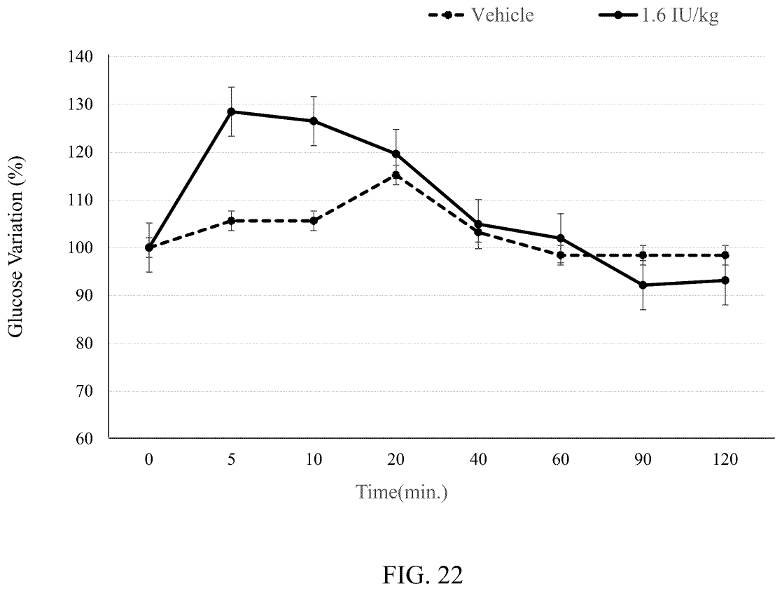

FIG. 22 shows data expressed as the percentage of variation in blood glucose levels relative to the basal levels (Time 0), after the transdermal application of human insulin solution (1.6 IU/kg/ml), or the vehicle (1 ml/kg). Statistical analysis was performed using one-way ANOVA test followed by Dunnett's multiple comparison test. Statistical significance is shown as * p<0.05.

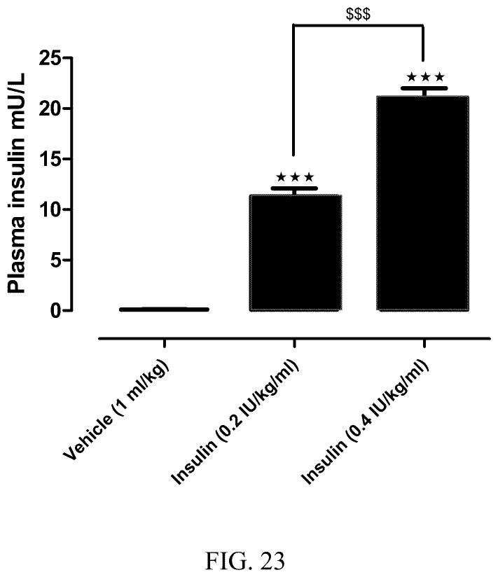

FIG. 23 shows human insulin levels measured in cumulative plasma samples collected from time 0 (before treatment to 120 min after treatment. Results are means+SE of 2 values for the vehicle treated rats and 4 values for the 0.2 IU and 0.4 IU/kg/ml treated animals. Statistical significance is shown as *** p<0.001, using one-way ANOVA test followed by Dunnett's multiple comparison test to the vehicle treated group. Statistical significance is shown as *** p<0.001, using Student's t-test to compare 0.2 IU, and 0.4 IU/kg/ml insulin treated groups.

### CONCLUSION

The above data showed that TD insulin has crossed the skin barrier and has been detected in rats' plasma in a dose-response manner.

The above data also demonstrates that the transdermal insulin delivery route has a decreased effect on fasted blood glucose in Wistar rats, when compared to the placebo or vehicle treated animals. Despite that we were not able to produce the data of human insulin time course after crossing the skin barrier, nevertheless cumulative human levels were detected in treated rat plasma samples. This confirms that the transdermal delivery system acts as a vector or carrier permitting human insulin to cross the skin barrier and thus to reduce blood glucose levels.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A transdermal insulin formulation comprising:
(i) insulin; and
(ii) a solvent system comprising one or more selected from the group consisting of at least two solvents, at least one solvent modifier, at least one solute modifier, at least one source of cellular activation energy, and at least one skin stabilizer;
wherein the solvent system comprises molecular properties substantially similar or approximately ±20% to molecular properties of the insulin within the formulation, said molecular properties selected from van der Waals forces and dipole moments.

2. The formulation according to claim 1, wherein:
(a) the at least two solvents are present in an amount ranging from about 5% wt/wt to about 90% wt/wt of the formulation,
(b) the at least one solvent modifier is present in an amount ranging from about 0.0001% wt/wt to about 50% wt/wt of the formulation,
(c) the at least one solute modifier is present in an amount ranging from about 0.003% wt/wt to about 5% wt/wt of the formulation,
(d) the at least one source of cellular activation energy is present in an amount ranging from about 0.01% wt/wt to about 0.1% wt/wt of the formulation, and
(e) the at least one skin stabilizer is present in an amount ranging from about 0.05% wt/wt to about 5% wt/wt of the formulation.

3. The formulation according to claim 1, wherein the solvent system further comprises one or more of selected from the group consisting of a membrane permeability modifier; an enzyme activator; and a capillary dilator.

4. The formulation according to claim 1, wherein the solvent system further comprises one or more of:
(a) a membrane permeability modifier in an amount ranging from about 0.01% wt/wt to about 5% wt/wt of the formulation,
(b) an enzyme activator in an amount ranging from about 0.01% wt/wt to about 0.05% wt/wt of the formulation, and
(c) a capillary dilator in an amount ranging from about 0.1% wt/wt to about 2% wt/wt of the formulation.

5. The formulation according to claim 1, wherein the insulin is selected from the group consisting of a rapid-acting insulin, a short-acting insulin, an intermediate-acting insulin, a long-acting insulin, and a mixture thereof.

6. The formulation according to claim 1, wherein the at least two solvents are selected from the group consisting of ethanol, propylene carbonate, propylene glycol, and acetone.

7. The formulation according to claim 16, wherein
(i) the ethanol is present in an amount ranging from 37% wt/wt to 54% wt/wt of the formulation:
(ii) the propylene carbonate is present in an amount ranging from 44% wt/wt to 58% wt/wt of the formulation;
(iii) the propylene glycol is present in an amount ranging from 40% wt/wt to 51% wt/wt of the formulation; and
(iv) the acetone is present in an amount ranging from 0.6% wt/wt to 4% wt/wt of the formulation.

8. The formulation according to claim 67, further comprising phosphoric acid or any other acid in an amount of the acid to insulin molecular ratio ranging from 0.2 to 2.0.

9. The formulation according to claim 1, wherein the formulation is formulated in a liquid dose form.

10. The formulation according to claim 1 in a liquid dosage form, wherein the liquid dosage form ranges from 0.2 mL to 1 mL and comprises insulin in an amount ranging from 7 IU/mL to 1,700 IU/mL.

11. The formulation according to claim 1, wherein the solvent system comprises one or more selected from the group consisting of lemon oil, vitamin E, phytantriol, dexpanthenol, monolaurate, methylsulfonylmethane (MSM), and forskolin.

12-15. (canceled)

16. The formulation according to claim 11, wherein
(iii) the lemon oil is present in an amount ranging from 0.1% wt/wt to 0.9% wt/wt of the formulation;
(iv) the vitamin E is present in an amount ranging from 0.005% wt/wt to 0.1% wt/wt of the formulation;
(v) the phytantriol is present in an amount ranging from 0.003% wt/wt to 0.5% wt/wt of the formulation;
(vi) the dexpanthenol is present in an amount ranging from 0.008% wt/wt to 0.5% wt/wt of the formulation;
(vii) the monolaurate is present in an amount ranging from 0.09% wt/wt to 0.5% wt/wt of the formulation;
(viii) the methylsulfonylmethane is present in an amount ranging from 0.1% wt/wt to 1% wt/wt of the formulation; and
(ix) the forskolin is present in an amount ranging from 0.01% wt/wt to 0.07% wt/wt of the formulation.

17. A process for making the formulation according to claim 1, the process comprising:
(a) selecting the insulin,
(b) determining an effective dose of the insulin, said effective dose of the insulin having molecular properties including van der Waals forces and dipole moments,
(c) quantifying said molecular properties of the insulin,
(d) determining an amount of the solvent system to solubilize said effective dose of the insulin, said amount of the solvent system having molecular properties including van der Waals forces and dipole moments,
(e) quantifying said molecular properties of said amount of the solvent system,
(f) comparing said molecular properties of the insulin and said molecular properties of the solvent system,
(g) adjusting the molecular properties of the solvent system so that they are substantially the same or approximately ±20% as the molecular properties of the insulin, and
(h) combining the solvent system and the insulin to provide the transdermal formulation.

18. A method of delivering insulin to a subject in need thereof, comprising administering to the subject a therapeutically effective amount of the transdermal formulation according to claim 1.

19. A method of stabilizing glucose levels in a subject receiving insulin, comprising administering to the subject in need thereof a therapeutically effective amount of the transdermal formulation according to claim 1.
